(19) United States
(12) Patent Application Publication
(10) Pub. No.: US 2026/0085338 A1
(43) Pub. Date: Mar. 26, 2026

(54) PSEUDOMONAS RECOMBINANT PROTEIN EXPRESSION SYSTEM

(71) Applicant: KATHOLIEKE UNIVERSITEIT LEUVEN, Leuven (BE)

(72) Inventors: Rob LAVIGNE, Ekeren (BE); Maarten BOON, Heverlee (BE); Eveline-Marie LAMMENS, Rotselaar (BE)

(21) Appl. No.: 19/109,298

(22) PCT Filed: Sep. 6, 2023
(86) PCT No.: PCT/EP2023/074505

§ 371 Date: Mar. 6, 2025

(30) Foreign Application Priority Data
Sep. 6, 2022 (EP) 22194203.0
Apr. 6, 2023 (EP) 23167047.2

(51) Int. Cl. C12P 21/00; C12N 9/12; C12N 15/78

(52) U.S. Cl. CPC C12P 21/00; C12N 9/127; C12N 15/78; C12Y 207/07048; C12N 2830/002

(57) ABSTRACT

The invention relates to a Pseudomonas sp. strain for use in the production of a recombinant protein characterised in that said strain comprises a nucleotide sequence encoding a phi15 RNA polymerase. The invention further relates to a plasmid, capable of integrating or replicating in Pseudomonas sp., comprising a phi15 promoter sequence operably linked to a nucleotide comprising one or more restriction sites for the insertion of a nucleotide sequence encoding a recombinant protein, or operably linked to a nucleotide sequence encoding a recombinant protein.

### CROSS-REFERENCE TO RELATED APPLICATIONS

This application is a National Stage Entry under 35 U.S.C. § 371 of International Application No. PCT/EP2023/074505, filed Sep. 6, 2023, and claims benefit of priority to European Application No. 22194203.0, filed Sep. 6, 2022, and European Application No. 23167047.2, filed Apr. 6, 2023, each of which is incorporated herein by reference in its entirety.

### SEQUENCE LISTING

A sequence listing in XML format having file name “Sequence_KAT0088PA.xml” (54,984 bytes), created on Jun. 27, 2025, is incorporated herein by reference.

### FIELD OF THE INVENTION

The invention relates to the expression of recombinant proteins in Pseudomonas bacteria.

### BACKGROUND OF THE INVENTION

Due to their long-standing co-evolution, bacteriophage genomes encode unique biological parts and cell modulators that are integrally adapted to their host, including viral RNA polymerases (RNAPs) and orthogonal promoters. The main example hereof is the transcriptional machinery of coliphage T7. Since its discovery over 30 years ago, the T7 transcription elements have become indispensable for both high-yield protein production in Escherichia coli and the construction of complex Synthetic Biology (SynBio) circuitry. The transcriptional machinery of T7, including its small, single subunit, RNAP, 17 bp promoter (PT7), and specific T7 lysozyme, has since been commercialized by Novagen (Merck Group, Germany) as the well-known pET system. The pET system consists of three modules and can be induced with isopropyl-β-D-thiogalactopyranoside (IPTG), which drives the expression of the T7 RNAP by the LacI expression system (FIG. 1). Next, the T7 RNAP transcribes any gene of interest from PT7 to extremely high levels, resulting in a high (recombinant) protein yield. Due to the strong activity of the T7 RNAP, strict regulation of this system is required to avoid the unwanted expression of the gene of interest in pre-production cultures. The presence of the T7 lysozyme supports this regulation, as it inhibits the T7 RNAP when expressed in low basal amounts prior to IPTG induction.

Apart from the traditional pET system for protein production, the T7 RNAP has several interesting properties which have been exploited in SynBio applications. First, the exceptional transcriptional activity of the T7 RNAP can be implemented in applications beyond protein production, such as signal amplification in biosensors and enzymatic activity assays. Second, the modular structure of the T7 RNAP allows the splitting of the T7 RNAP gene into two parts to create AND and OR gates in synthetic circuits. In addition, the separate T7 RNAP modules can be fused to other enzymes, such as deaminases, to enable base-editing of a target sequence. Third, the T7 RNAP solely recognizes P_{T7} and can, therefore, function fully orthogonally from the host's transcriptional machinery.

In the past decade, several bacterial species beyond E. coli have emerged as valuable hosts for biotechnological and SynBio applications. In specific cases, non-model bacteria are better tailored to produce certain proteins or metabolites due to their unique metabolism, ability to thrive in harsh bioreactor setups, and/or intrinsic resistance to toxic proteins and metabolites that are industrially relevant. These species include members of the Pseudomonas genus, with a special focus on the metabolically versatile and robust P. putida [Loeschcke et al. (2015) Appl. Microbiol. Biotechnol. 99, 6197-6214; Kivisaar (2020) J. Med. Microbiol. 69, 324-338] As such, SynBio parts and tools, including a high-yield expression system, for these hosts are needed. The powerful pET system would be a great addition to the Pseudomonas toolbox but performs sub-par in Pseudomonas hosts as it is based on coliphage T7 and is further optimized towards E. coli. In P. putida, induction of the T7 RNAP and T7 lysozyme results in arrested cell growth, leading to high cell burden and increased mutational pressure [Shis et al. (2013) Proc. Natl. Acad. Sci. USA 110, 5028-5033; Kushwaha (2015) Nat. Commun. 6, 7832; Beentjes et al. (2022) ACS Synth. Biol. 11, 3939-3953; Liang et al. (2018) ACS Synth. Biol. 7, 1424-1435; Weihmann et al. (2019) Microb. Biotechnol. 13, 250-262]. Moreover, the LacI system, which is often used to express the T7 RNAP, is regulated differently in P. putida and causes extremely high levels of T7 RNAPs under uninduced conditions [Calero et al. (2016) ACS Synth. Biol 5, 741-753; Cook et al. (2018) J. Ind. Microbiol. Biotechnol. 45, 517-527; Martin-Pascual et al. (2021) Biotechnol. Adv. 49, 107732]. In the past, attempts have been made to solve these issues by employing the XyIS/Pm system instead of LacI [Herrero et al. (1993) Gene 134, 103-106; Liang et al. (2021) Front. Chem. 9, 1-8] or by using antisense RNA to tightly regulate the system. However, these systems still rely on the toxic T7 RNAP. Furthermore, Liang et al. cited above reported a functional alternative RNAP for P. putida but did not report on the fitness of the expression strains. As a result, the pET system is rarely used in P. putida, and researchers instead rely on the established XyIS/Pm and RhaRS/P_{rhaBAD} expression systems for protein production. These expression systems are generally well-regulated and characterized in P. putida but lack the extremely high transcription levels enabled by the T7 RNAP.

### SUMMARY OF THE INVENTION

The present invention characterizes four alternative RNAPs from Pseudomonas phages that exhibit a broad range of expression levels, do not show any toxicity to their host, and operate orthogonally to each other and the T7 RNAP. Efficient expression of these RNAPs was achieved using the XyIS/Pm system, and tight regulation was improved by introducing the corresponding phage lysozymes. The phage T7 RNA polymerase and lysozyme form the basis of the widely used pET expression system for recombinant expression in the biotechnology field and as a tool in microbial synthetic biology. Attempts to transfer this genetic circuitry from Escherichia coli to non-model bacterial organisms with high potential have been restricted by the cytotoxicity of the T7 RNAP in the receiving hosts. We here explore the diversity of T7-like RNAPs mined directly from Pseudomonas phages for implementation in Pseudomonas species, thus relying on the co-evolution and natural adaptation of the system towards its host. By screening and characterizing different viral transcription machinery using a vector-based system in P. putida., we identified a set of four non-toxic phage RNAPs from phages phi15, PPPL-1, Pf-10, and 67PfluR64PP, showing a broad activity range and orthogonality to each other and the T7 RNAP. In addition, we confirmed the transcription start sites of their predicted promoters and improved the stringency of the phage RNAP expression systems by introducing and optimizing phage lysozymes for RNAP inhibition. This set of viral RNAPs expands the adaption of T7-inspired circuitry towards Pseudomonas species and highlights the potential of mining tailored genetic parts and tools from phages for their non-model host.

Besides the excellent performance of the phi15 expression system in vivo in P. putida, the phi15 RNAP host and plasmids vector series can be employed in in vitro transcription. In particular, purified phi15 RNAP enzyme can yield high transcriptional levels of a target sequence on a pPUT vector or alternative (linear) polynucleotide preceded by the phi15 promoter sequence.

Besides production of recombinant protein, the phi15 RNAP can be used for expression of a reporter protein. By coupling the induction of phi15 RNAP to a specific trigger, a biosensor system is created, where the trigger will induce phi15 RNAP expression, which will generate high amounts of the reporter. This way, a very sensitive system with a low detection limit can be constructed.

The invention is further summarised in the following statements:
1. A Pseudomonas sp. strain for use in the production of a recombinant protein characterised in that said strain comprises a nucleotide sequence encoding a phi15 RNA polymerase.
2. The Pseudomonas sp. strain according to statement 1, wherein said phi15 RNA polymerase under the control of an inducible promotor.

“Phi15 RNA polymerase” encompasses the protein with accession number YP_004286187.1, as well polymerase proteins with an amino acid sequence which is more than 95%, more than 98% or more than 99% identical to this Phi15 RNA polymerase, and having RNA polymerase activity. It equally encompasses truncated versions of this protein at the N-terminus and/or C-terminus which maintain RNA polymerase.

A particular modified Phi15 RNA po “ymer'se Is Phi15 RNA polymerase with the R630S mutation as indicated in the section with protein and DNA sequence.
3. The Pseudomonas sp. strain according to statement 1 or 2, wherein said phi15 RNA polymerase has the sequence of the protein of accession number YP_004286187.1.
4. The Pseudomonas sp. strain according to any one of statements 1 to 3, wherein said phi15 RNA polymerase has a R630S mutation with reference of the sequence of Phi15 RNA polymerase YP_004286187.1.
5. The Pseudomonas sp. strain according to any one of statements 2 to 4, wherein the inducible promotor is the XyIS/pM promoter/regulator.

Other suitable promoter/regulator systems are for example LacI/PlacUV5, RhaRS/prhaBAD en AraC/paraBAD.

An inducible promoter/regulator has the advantage to switch the production of recombinant protein ON or OFF according to the need of the user. In addition, the level of protein production can be altered by increasing or reducing the inducer concentration.

In contrast therewith a constitutive promoter does not require the use of a specific inducer and reduces the cost.
6. The Pseudomonas sp. strain according to any one of statements 1 to 5, wherein said nucleotide sequence is integrated in the genome of said Pseudomonas strain.

While the nucleotide sequence encoding the phi15 RNA polymerase and other nucleotide sequence encoding proteins sequences for use in the expression system of the present invention can be provided on a plasmid, integration of these nucleotide sequence overcomes the need of additional selection markers to maintain such plasmids in the bacterial host.
7. The strain according to any one of statements 1 to 6, wherein said nucleotide sequence is integrated in the PP13 (gyrB) locus of said Pseudomonas sp. strain.

It is shown in the examples of the present invention that integration in this region leads to the highest expression of the RNA polymerase Other suitable places in the Pseudomonas genome for integration are for example the loci PP5322 or PP5042. The numbering of the loci refers to the annotation of the genome of P. putida KT2440, and is known to the skilled person. The loci can be easily identified in other Pseumonas_strains.
8. The strain according to any one of statements 1 to 7, wherein said Pseudomonas sp. is Pseudomonas putida.

Another suitable Pseudomonas sp is e.g. Pseudomonas aeruginosa or Pseudomonas chlororaphis.
9. The strain according to any one of statements 1 to 8, wherein said Pseudomonas sp. is Pseudomonas putida strain KT2440.
10. The strain according to any one of statements 1 to 9, wherein the ribosome binding sequence for the translation of the phi15 RNA polymerase is the weak RBS with sequence TAAAGCTTTATCTATTTAACAACGCGGTCCGATGTG [RBC-C] [SEQ ID NO: 1] or with sequence TAAAGCTTTATCTATTTAACAACGGGGTCCGAGGTG [RBS-D] [SEQ ID NO:2].

The use of such a weaker RBS has the advantage that the basal expression levels of phi15 RNAP in uninduced conditions are significantly reduced, which 1) reduces leakiness/basal expression of the recombinant protein in uninduced conditions and 2) improves the dynamic range of the expression system.
11. The strain according to any one of statements 1 to 10, further comprising a nucleotide sequence encoding a lysozyme.
12 The strain according to statement 11, wherein the lysozyme is phi15 lysozyme with accession number YP_004286199.1.

Another suitable lysozyme is for example Pf-10 lysozyme.

“Lysozyme” encompasses the wt protein as defined by its accession number (YP_004286187.1 for phi15 lysozyme, as well lysozyme proteins with an amino acid sequence which is more than 95%, more than 98% or more than 99% identical to this lysozyme, and having RNA polymerase inhibition. It equally encompasses truncated versions of this protein at the N-terminus and/or C-terminus which maintain RNA polymerase inhibition.
13. The strain according to statement 11 or 12, wherein the nucleotide sequence encoding said lysozyme is integrated in the genome of said Pseudomonas sp.
14. The strain according to any one of statements 11 to 13, wherein the sequence encoding said lysozyme is integrated in locus PP4305 the genome of Pseudomonas putida (typically strain KT2440).

Another suitable site for lysozyme integration is e.g. PP5388. Also here the numbering of the loci refers to the annotation of the genome of P. putida KT2440.
15. The strain according to statement 11 or 12, comprising a plasmid vector comprising the nucleotide sequence encoding said lysozyme. Other suitable lysozymes are for example pf-10 lysozyme.
16. The strain according to any one of statements 11 to 15, wherein said lysozyme is under the control of the an inducible promotor.
17. The strain according to statement 16, wherein said promotor is the RhaRS/P_{rhaBAD} promotor/regulator.
18. The strain according to any one of statements 11 to 15, wherein the sequence encoding a phi15 lysozyme is under the control of a constitutive promoter.
19. The strain according to statement 18, wherein said constitutive promoter is p14c.
20. The strain according to any one of statements 11 to 19, wherein said lysozyme is the G3RQ mutant of phi15 lysozyme, wherein glycine at position 3 is replaced by the dipeptide arginine-glutamine.

This mutant lysozyme significantly reduced leakiness of phi15 RNA polymerase. Other useful mutants of ph15 lysozyme in this context are GR3, G3Q, K5Q.
21. The strain according to any one of statements 11 to 20, wherein the nucleotide sequence encoding said lysozyme comprises the BCD22 ribosome binding site. Other suitable RBS for lysozyme are e.g. BCD13 or BCD2. These RBS are disclosed in Mutalik et al. (2013) Nature Methods 10, 354-360.
22. The strain according to any one of statements 1 to 21, further comprising a nucleotide sequence encoding the Phi15 GP16 RNA polymerase inhibitor with accession number YP_004286194.1.

“Phi15 GP16 RNA polymerase inhibitor” encompasses the protein with accession number YP_004286194.1, as well proteins with an amino acid sequence which is more than 95%, more than 98% or more than 99% identical to this polymerase inhibitor, and having RNA polymerase inhibitor activity. It equally encompasses truncated versions of this protein at the N-terminus and/or C-terminus which maintain RNA polymerase inhibitor activity.

The presence of Phi15 GP16 RNA allows to inhibit the replication of the bacterial host while the recombinant protein production remains active.

This RNA polymerase inhibitor can be located on a plasmid, or be integrated in the host genome.

The RNA polymerase is typically under the control of an inducible promotor, which may be the same one or a different one than the used to initiate recombinant protein expression.

In a specific embodiment the same inducible promotor is used to synchronise recombinant protein expression and host replication inhibition.

The presence of Phi15 GP16 RNA allows to inhibit the replication of the bacterial host while the recombinant protein production remains active.
23. A plasmid, capable of integrating or replicating in Pseudomonas sp., comprising a phi15 promoter sequence operably linked to a nucleotide comprising one or more restriction sites for the insertion of a nucleotide sequence encoding a recombinant protein, or operably linked to a nucleotide sequence encoding a recombinant protein.

Oris for Pseudomonas for single copy, medium copy or high copy replications are known in the art
24. The plasmid according to statement 23, comprising two transcriptional terminators sequences 3′ of said one or more restriction sites or 3′ of the nucleotide sequence encoding the recombinant protein.

This allows efficient termination of the phi15 RNAP, to avoid unwanted readthrough of downstream sequences. In addition, the terminator stabilizes the mRNA molecule and thus increases recombinant protein expression levels.
25. The plasmid according to statement 23 or 24, comprising a transcriptional terminator sequences 5′ of the phi15 promoter sequence.

This avoids unwanted readthrough of upstream coding regions into the expression construct.
26. The plasmid according to statement 25, wherein the transcriptional terminator sequence is LUZ7T50.
27. The plasmid according to statement 25, wherein the transcriptional terminator sequence is a combination of phi15 T5 and phi15 T1.
28. The plasmid according to any one of statements 23 to 27, wherein the plasmid comprises, 5′ of the RBS and 3′ of the MCS of the sequence encoding the recombinant protein, a further nucleotide sequence comprising an additional RBS and encoding an additional polypeptide sequence, thereby providing a bicistronic expression

A bicistronic design has the advantage that translation of the leader peptide will dissolve any secondary mRNA structures between the second RBS and coding region of the recombinant protein, thus ensuring efficient translation independent of the nucleotide sequence of the recombinant protein.
29. The plasmid according to statement 28, wherein said further nucleotide sequence has the sequence

[SEQ ID NO: 3]
GACAGAGGGAGCCGTTCCGTGCTCCTTCTGGACCCAATTTCTGACTCAAG
GAGAACTACATATGGCAACTCTGAACAACGGCACCAAGCAAGGCCAGAAT
AAGGAGGTTTTCTAATG

and encodes the amino acid sequence MATLNNGTKQGQNKEVF [SEQ ID NO:4].

Herein the underlined sequence is the ORF of the peptide MATLNNGTKQGQNKEVF [SEQ ID NO: 4].

Herein the ATG in bold is the startcodon of the recombinant protein.
30. A kit for the expression of recombinant protein comprising a host strain according to any one of statements 1 to 22 and comprising a plasmid according to any one of statements 23 to 29.
31. A host strain according to any one of statements 1 to 23 comprising a plasmid according to any one of statements 23 to 29.
32. A method of producing a recombinant protein, comprising the step of administering to a strain according to statement 31, an agent inducing the transcription and translation of the phi15 RNA polymerase.
33. The method according to statement 32, wherein the phi15 RNA polymerase is under the control of the XyIS/Pm regulator/promoter, and the inducing agent is 3-methylbenzoate.
34. Use of a strain according to any one of statements 1 to 22, or use of a plasmid according to any one of statements 23 to 29, or use of the strain according to statement 31, in a process of expressing a recombinant protein.
35. The use of a plasmid according to any one of statements 23 to 29, for in vitro transcription and/or translation of a target sequence.

### DETAILED DESCRIPTION

### Figure Legends

FIG. 1. General layout of the T7-based pET system for E. coli.

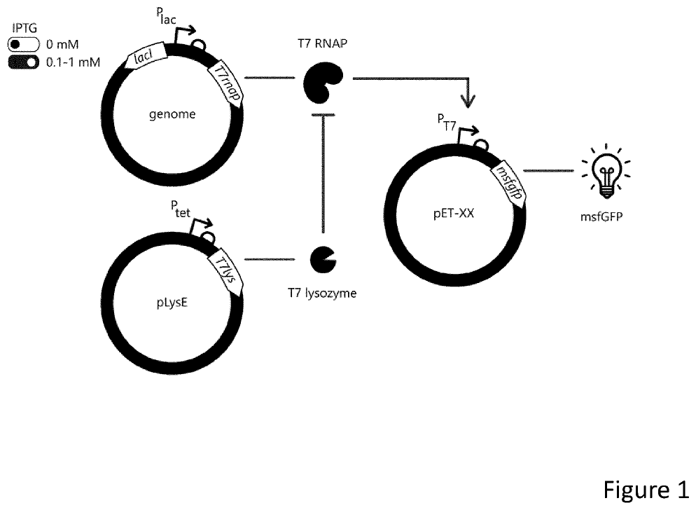

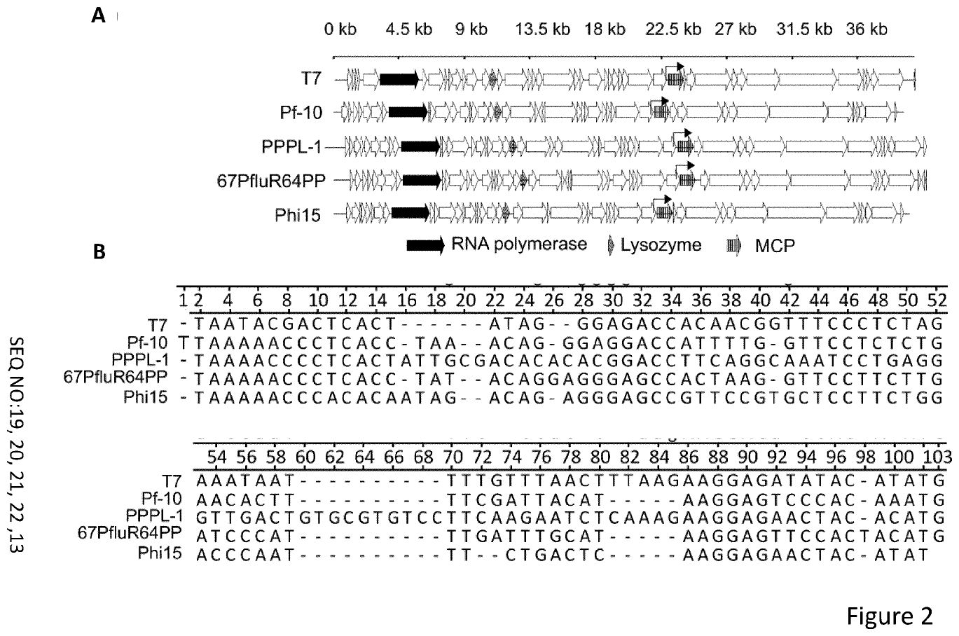

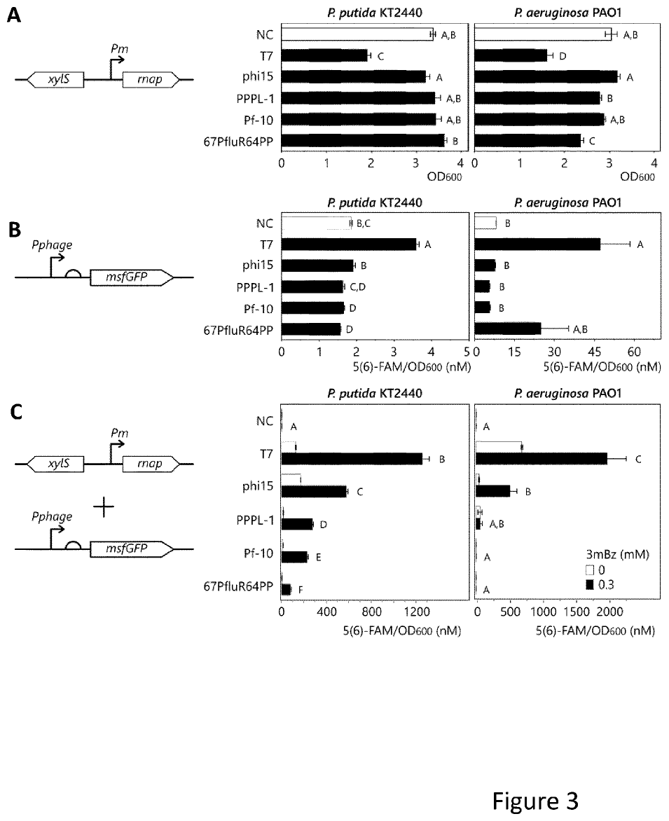

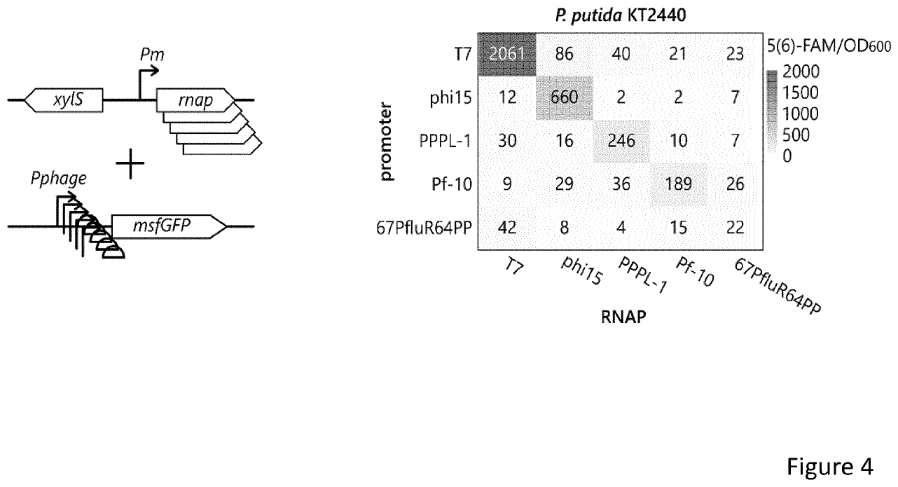

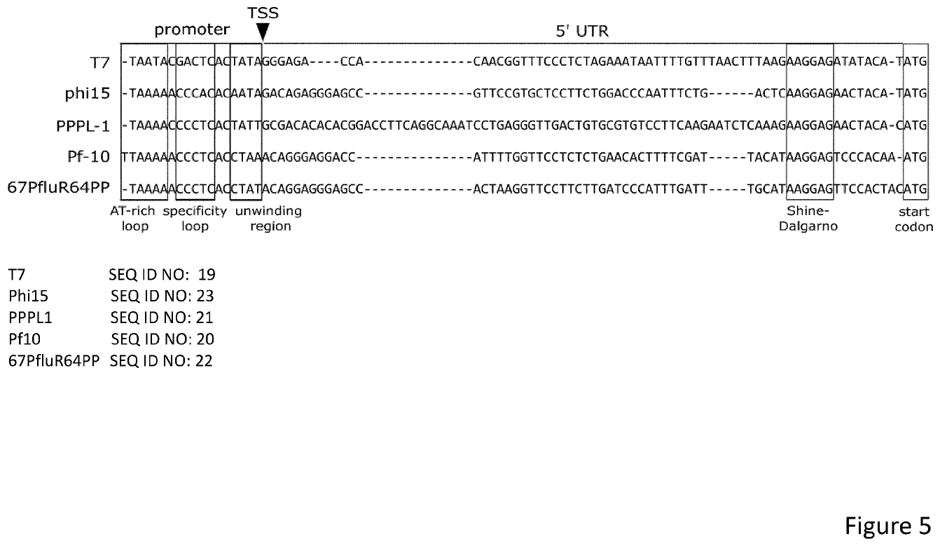

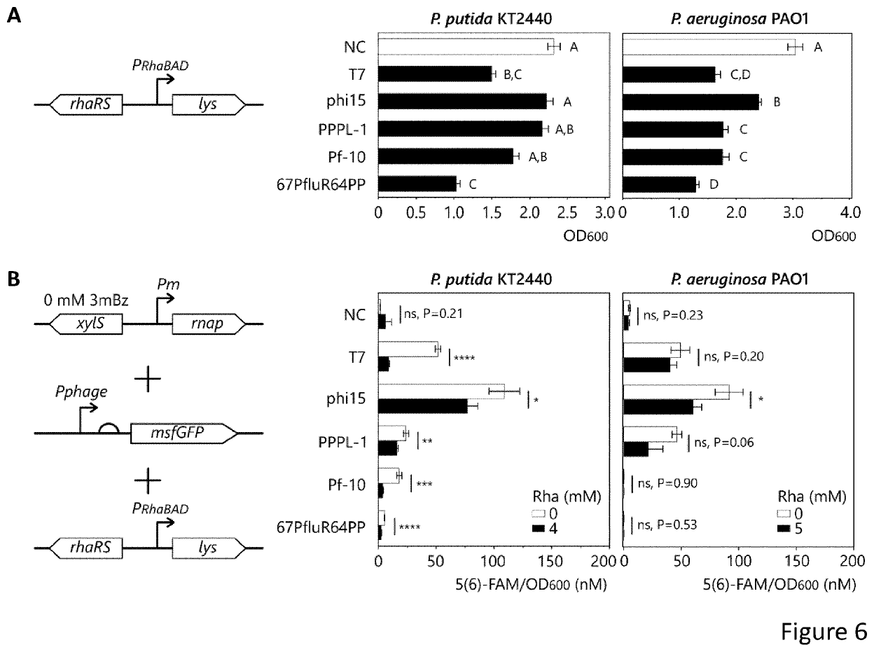

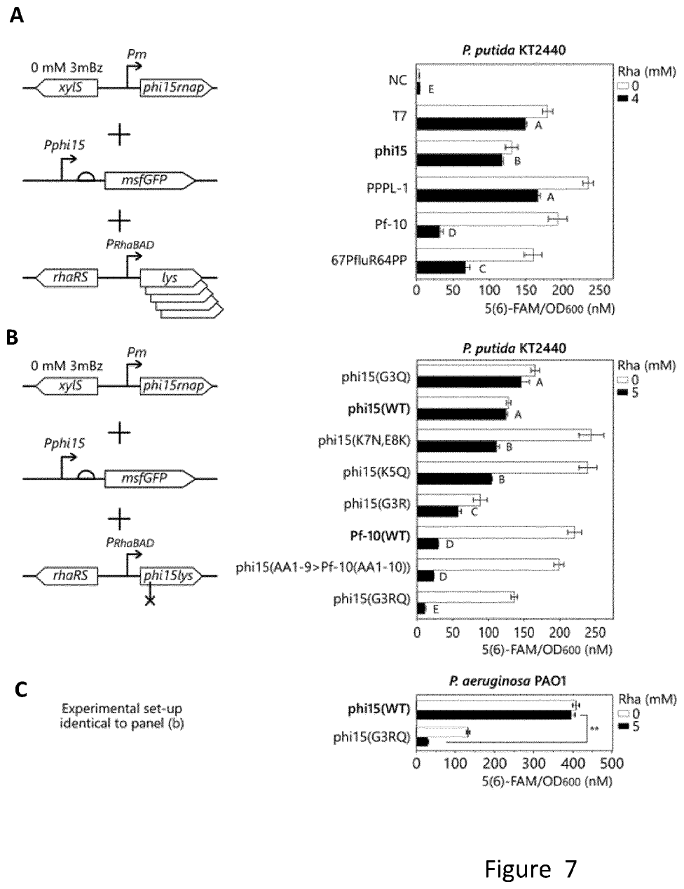

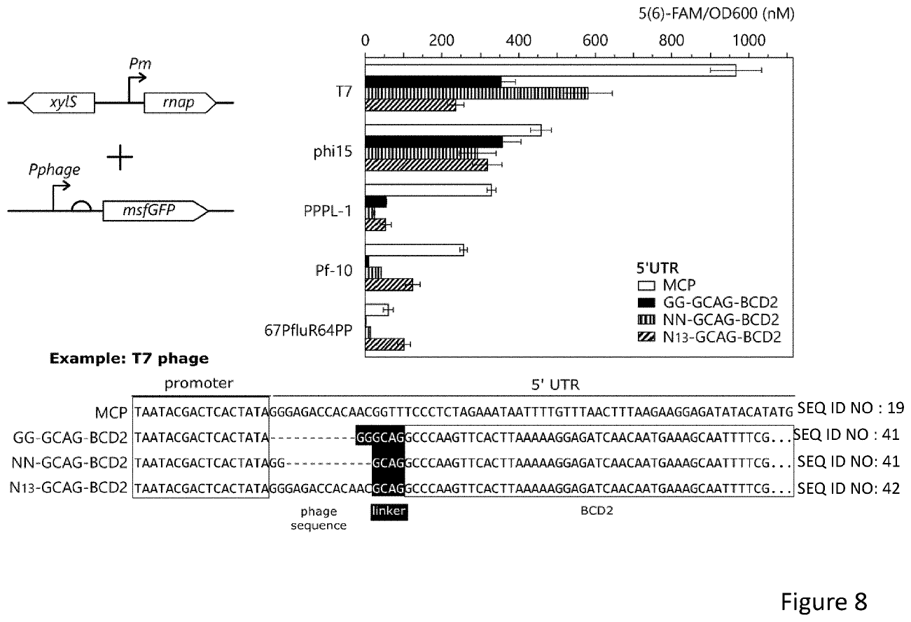

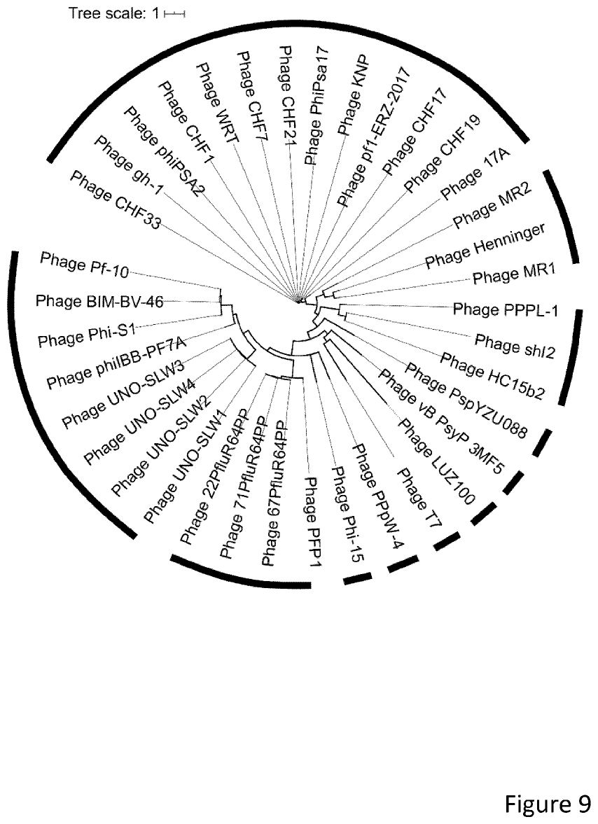

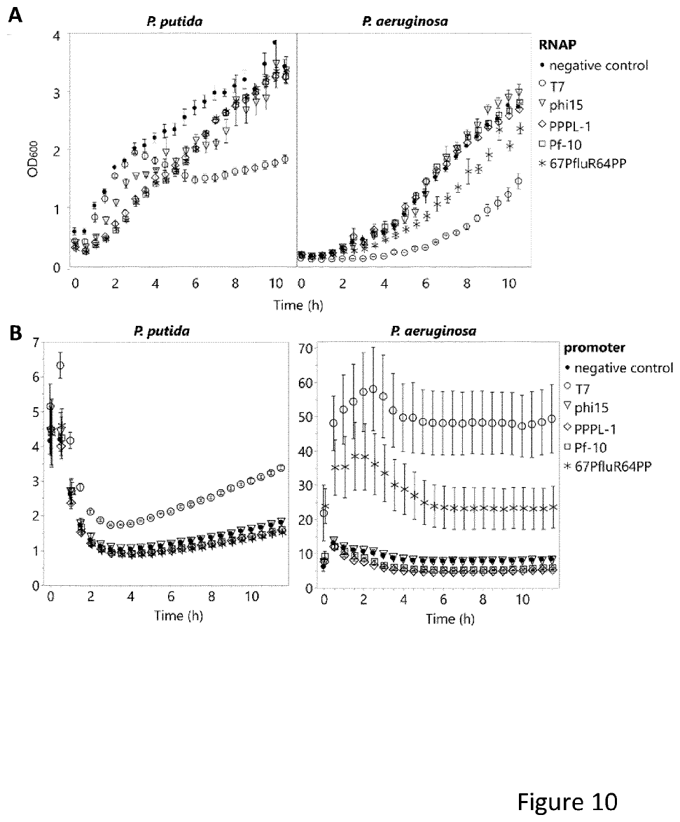

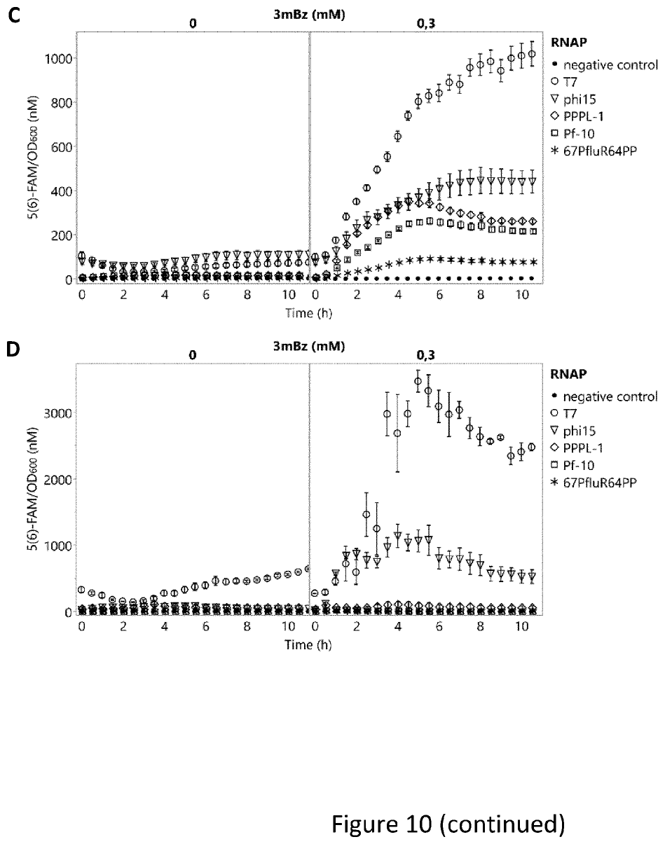

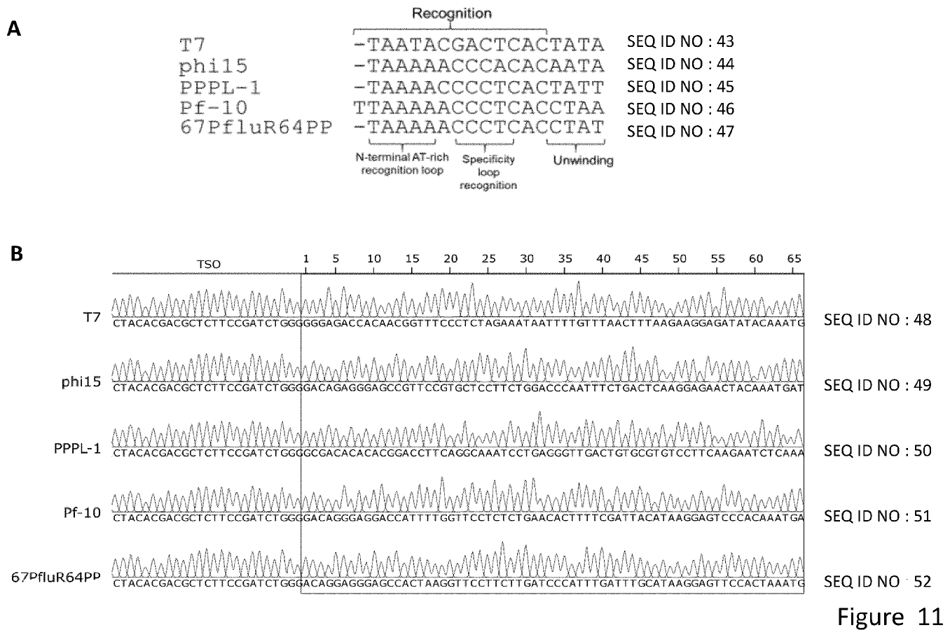

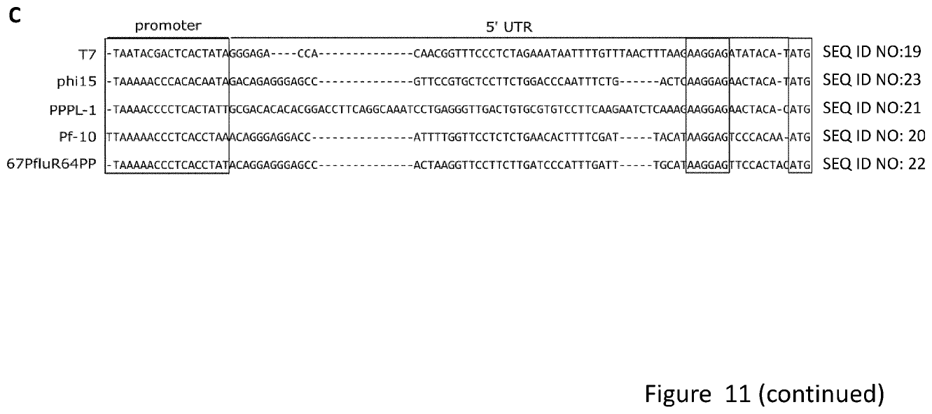

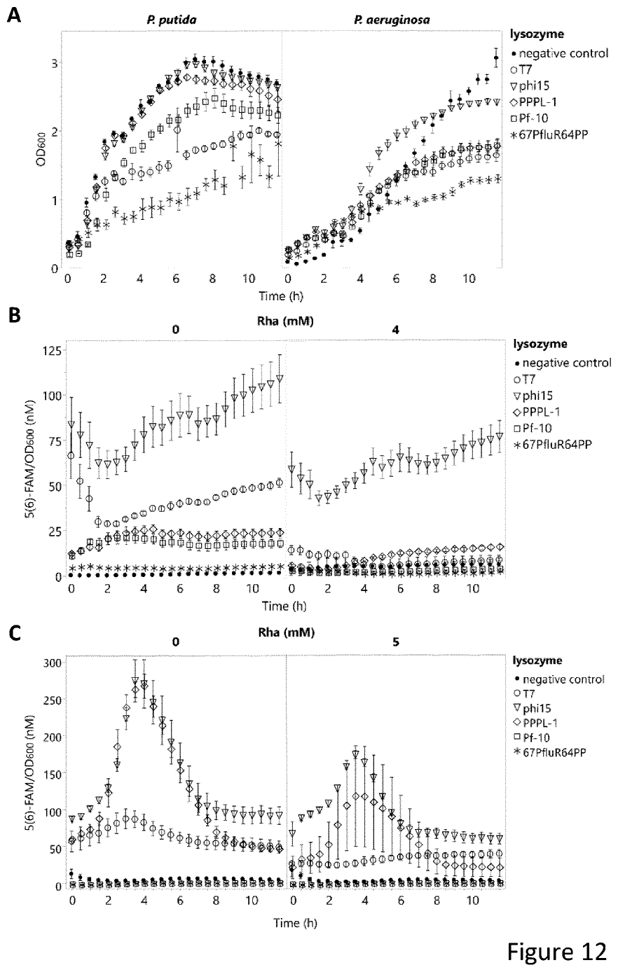

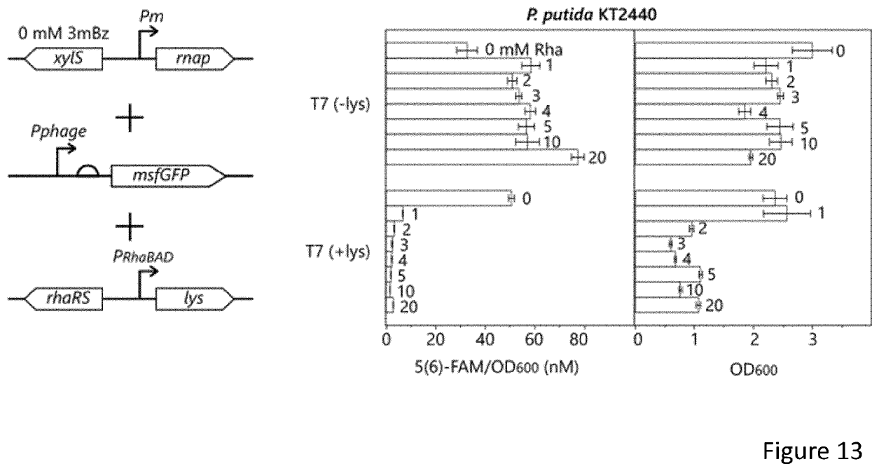

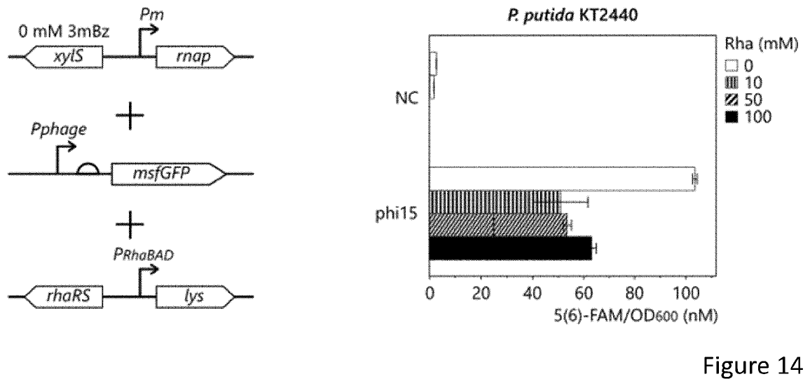

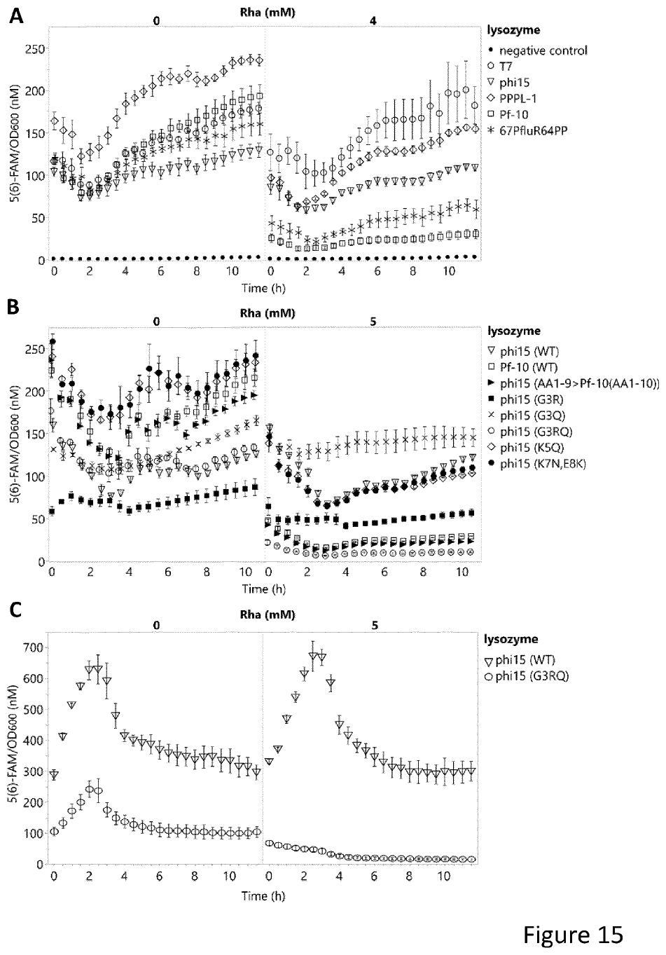

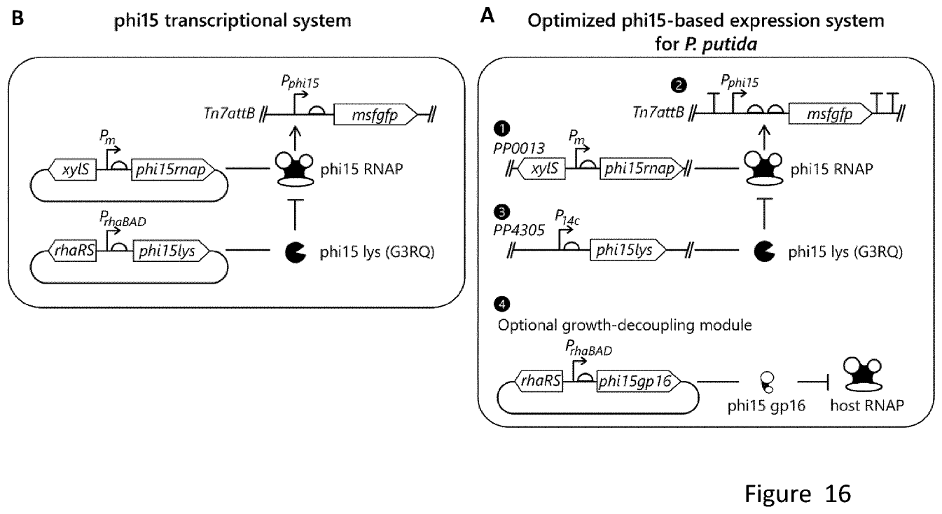

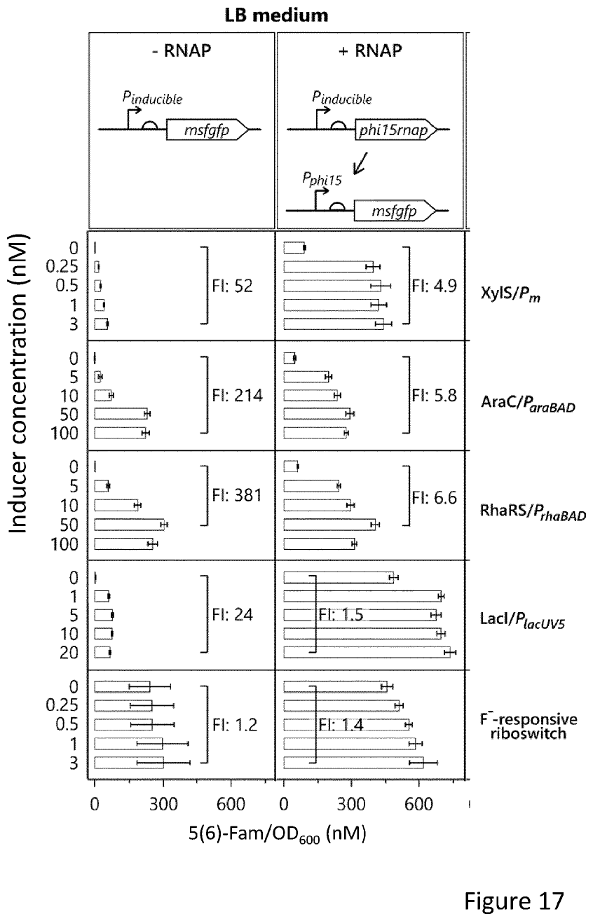

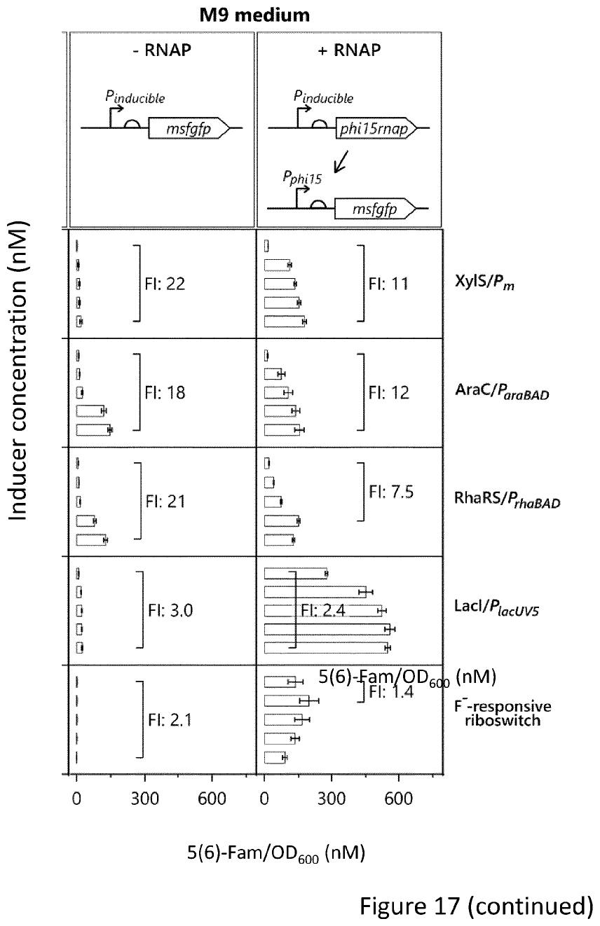

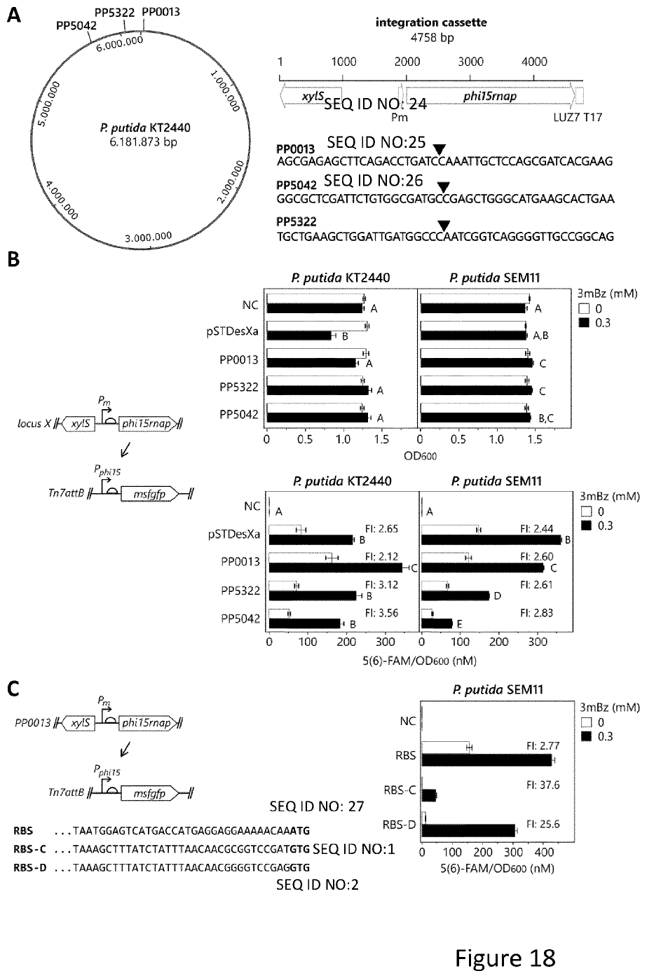

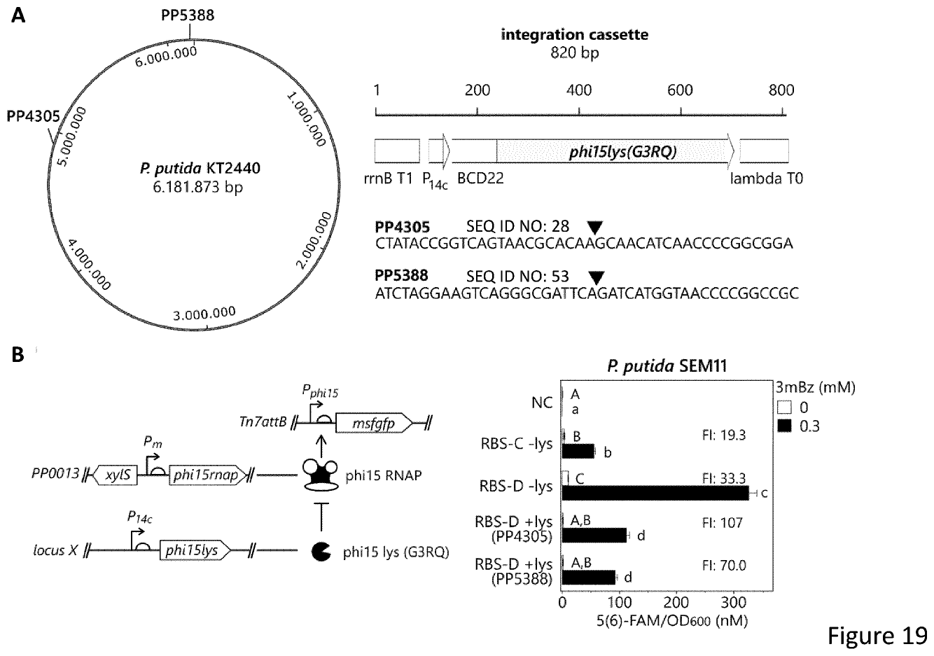

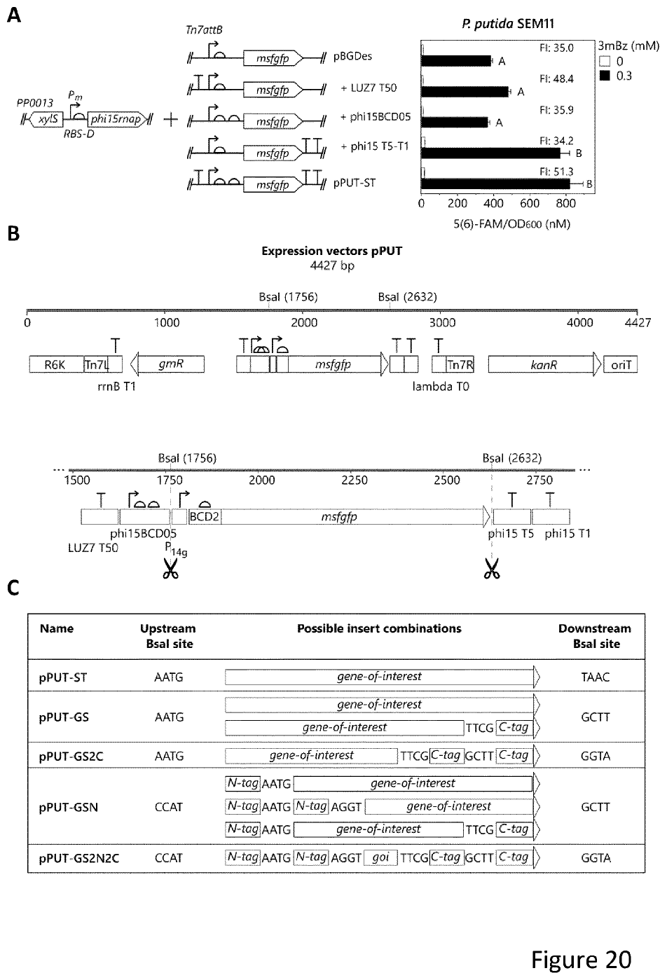

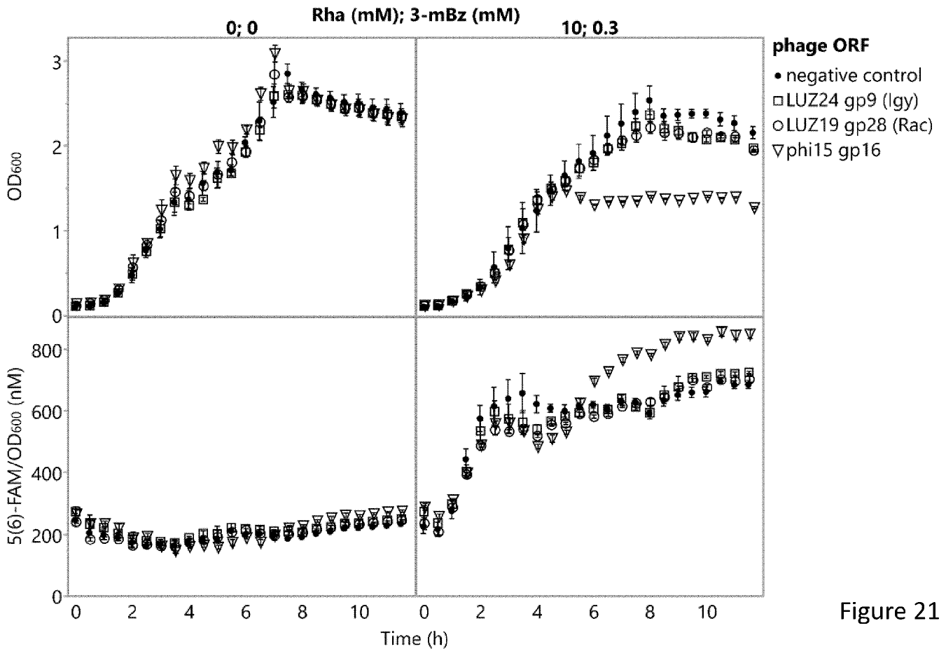

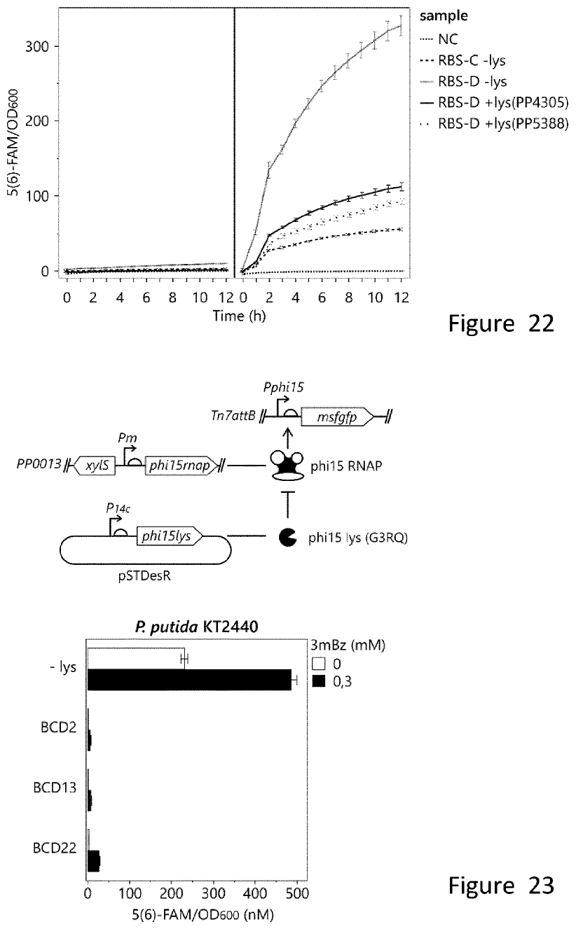

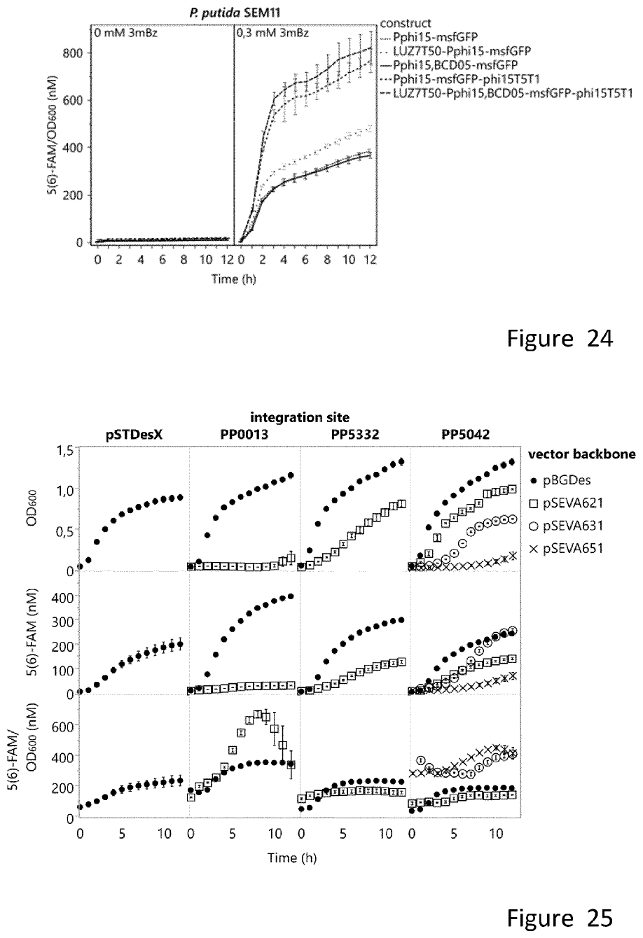

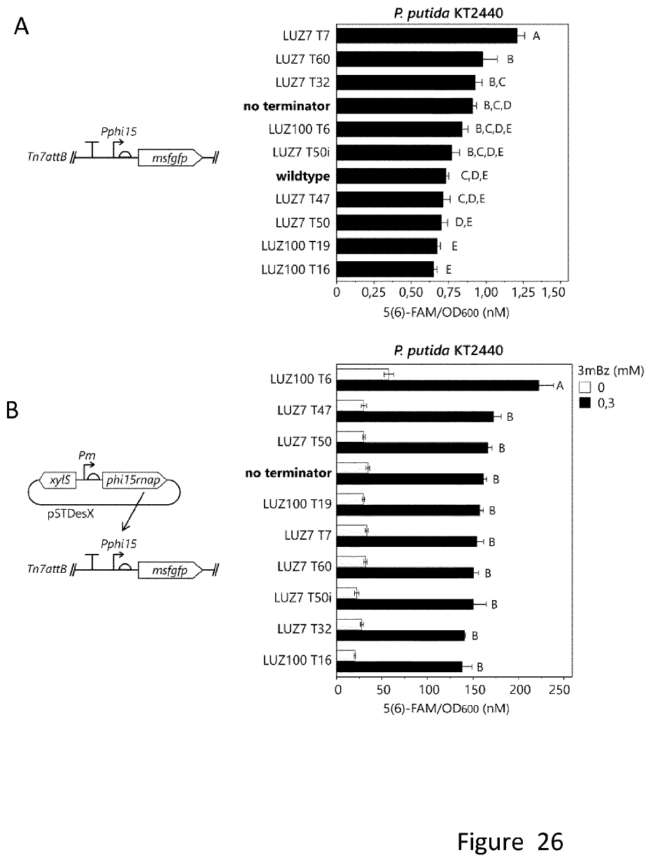

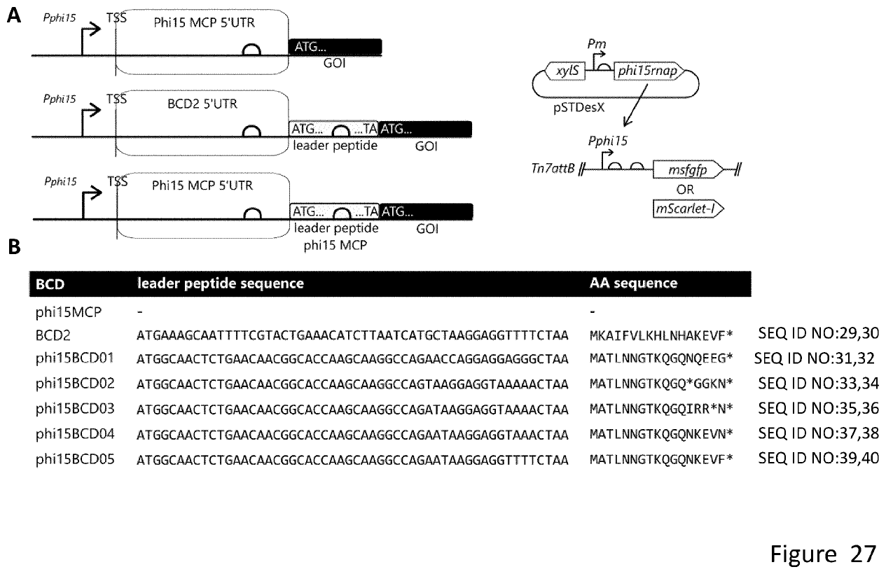

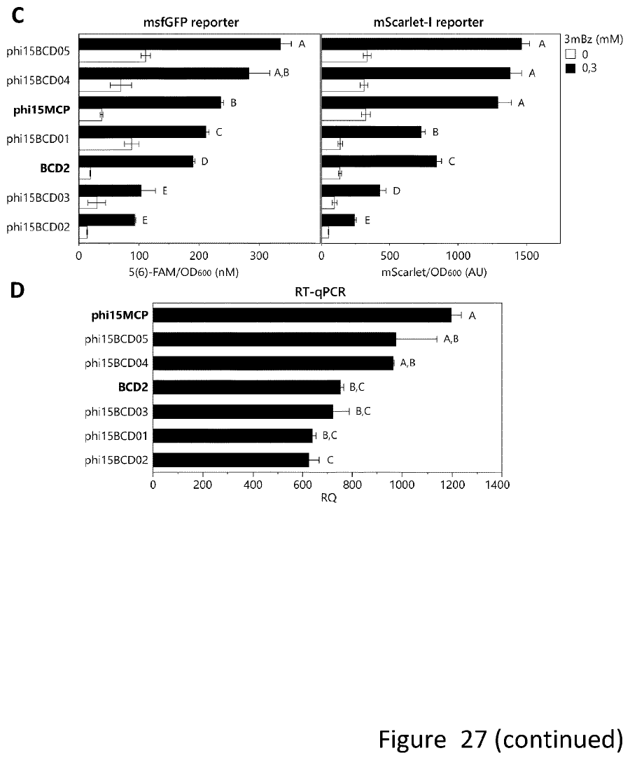

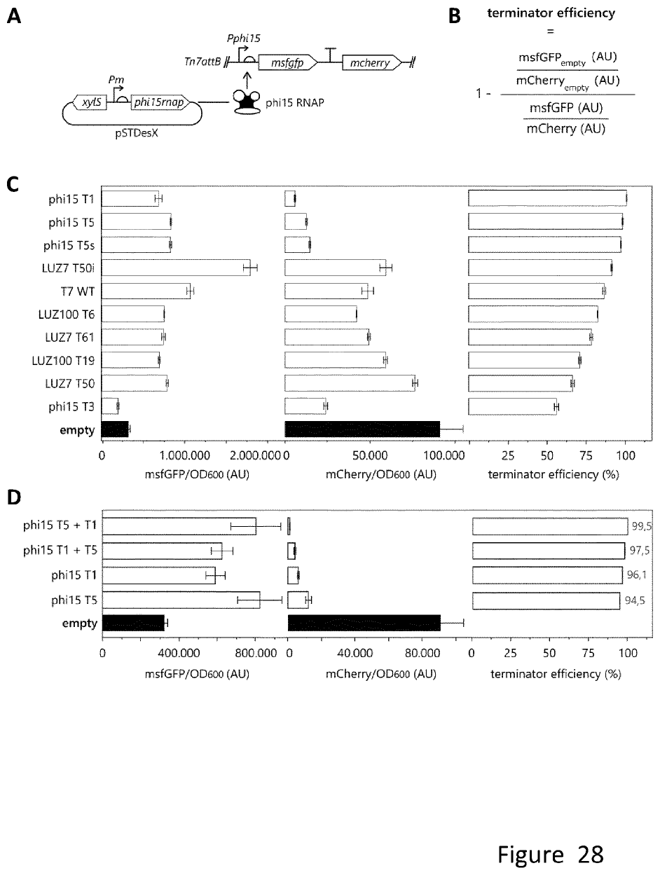

The T7 RNAP is stably integrated into the host genome with an IPTG-inducible expression cassette. In the absence of IPTG, the system is considered off, while upon addition of IPTG, usually 0.1-1 mM IPTG, the T7 RNAP is expressed from the LacI/Plac system. The T7 RNAP drives expression of a gene of interest, here depicted as an msfGFP, from its putative T7 promoter on any pET vector. To express toxic proteins, alternative hosts are available, carrying the pLys vector for expression of the T7 lysozyme, which inhibits transcriptional activity of the T7 RNAP in uninduced conditions. IPTG: isopropyl-β-D-thiogalactopyranoside, T7 RNAP: T7 RNA polymerase, msfGFP: monomeric superfolder green fluorescent protein.

FIG. 2. Genomic organization of phages T7, Pf-10, PPPL-1, 67PfluR64PP, and phi15.
A. The RNA polymerase genes, lysozyme genes, and major capsid protein (MCP) genes are indicated in black, dotted and striped arrows, respectively.
B. Multiple sequence alignment (ClustalOmega) of the predicted phage promoter and 5′ untranslated region of the phage's major capsid protein.

FIG. 3. Effect of phage RNAP expression on cell growth of P. putida KT2440 and P. aeruginosa PAO1.
A. The RNAPs of phages T7, phi15, PPPL-1, Pf-10, and 67PfluR64PP were introduced in P. putida and P. aeruginosa, as well as an empty pSTDesX vector as a negative control (NC). All samples were induced with 1 mM 3 mBz at OD₆₀₀ 0.3 for 12 h. Bars and error bars represent the mean OD₆₀₀ and standard error of four biological replicates after 12 h induction, respectively. Samples not connected by the same letters are significantly different (Tukey HSD, α=0.05).
B. Recognition of the phage promoter by the host RNAP of P. putida KT2440 and P. aeruginosa PAO1. MsfGFP reporter constructs with the phage-specific promoters of T7, phi15, PPPL-1, Pf-10, and 67PfluR64PP and a promoterless construct (NC) were introduced in P. putida or P. aeruginosa and were monitored for 12 h for OD₆₀₀₆₀₀ and msfGFP levels. As the phage RNAPs were not present, no msfGFP expression was expected unless the phage promoter was also recognized by the host RNAP. Bars and error bars represent the mean value and standard error of four biological replicates after 12 h cell growth, respectively. Samples not connected by the same letters are significantly different (Tukey HSD, α=0.05).
C. T7-like phage RNAPs generate high msfGFP expression levels from their putative phage promoter in P. putida KT2440 and P. aeruginosa PAO1. All phage RNAPs and corresponding phage promoter-msfGFP reporter constructs, including empty control vectors (NC), were introduced in P. putida and P. aeruginosa and were induced with 0.3 mM 3 mBz for a 12 h period. The fluorescent intensity was normalized for OD and expressed as an equivalent 5(6)-FAM concentration (nM). Bars and error bars represent the mean value and standard error of four biological replicates after 12 h induction, respectively. Samples not connected by the same letters are significantly different (Wilcoxon (P. putida) and Student's t-test (P. aeruginosa), α=0.05).

FIG. 4. Verification of cross-recognition between the phage RNAPs and their promoters.

All 25 combinations of RNAPs and Phage promoter-msfGFP reporter constructs were introduced in P. putida KT2440 and induced with 0.3 mM 3 mBz overnight. The fluorescent intensity was normalized for the OD and expressed as an equivalent 5(6)-FAM concentration (nM). Values represent the mean normalized fluorescence intensity after overnight induction of four biological replicates.

FIG. 5. Clustal-omega alignment of the validated promoter and 5′ UTR of the phages' major capsid protein, separated by the confirmed transcription start site (TSS).

Confirmed promoter regions of the T7 promoter and 5′ UTR, namely the AT-rich recognition loop, specificity loop, unwinding region, Shine-Dalgarno sequence, and start codon, are projected on the promoters of the other phages.

FIG. 6. Toxicity assay of phage lysozymes in P. putida KT2440 and P. aeruginosa PAO1.
A. All phage lysozymes of T7, phi15, PPPL-1, Pf-10, and 67PfluR64PP, as well as an empty pSTDesR control vector (NC), were introduced in P. putida and P. aeruginosa and were induced with 10 mM Rha at OD₆₀₀ 0.3, after which cell growth was monitored for 12 h. Bars and error bars represent the mean and standard error of four biological replicates. Samples not connected by the same letters are significantly different (Tukey HSD, α=0.05).
B. Fluorescence assay to analyse the inhibitory effect of the phage lysozyme on its corresponding phage RNAP. All phage RNAPs, lysozymes, and phage promoter-msfGFP reporter constructs of T7, phi15, PPPL-1, Pf-10, and 67PfluR64PP and empty control vectors (NC) were introduced in P. putida and induced with 4-5 mM Rha for 12 h. Data points represent the mean 5(6)-FAM/OD₆₀₀ value of four biological replicates, while error bars represent the standard error. Significance levels are indicated for one-sided Student's t-tests.

FIG. 7. Fluorescence intensity assay to analyse the inhibitory effect of different phage lysozymes on the phi15 RNAP.
A. The phage lysozymes of T7, phi15, PPPL-1, Pf-10, 67PfluR64PP, and an empty pSTDes3 control vector (NC) were introduced in P. putida KT2440 together with the phi15 RNAP and Pphi15-msfGFP reporter construct. All samples were induced with 4 mM Rha at OD₆₀₀0.3, after which the fluorescence intensity and cell growth were monitored every half hour for 12 h. Bars and error bars represent the mean 5(6)-FAM/OD₆₀₀ value and standard error of four biological replicates, respectively. Samples not connected by the same letters are significantly different (Tukey HSD, α=0.05).
B. Similar fluorescence assay as A. to assess the inhibitory strength of different phi15 lysozyme mutants on the phi15 RNAP. The wild-type phi15 and Pf-10 lysozymes (WT), as well as phi15 lysozyme mutants (AA1-9>Pf10 (AA1-10)), (G3R), (G3Q), (G3RQ), (K5Q), and (K7N,E8K), were introduced in P. putida, together with the phi15 RNAP and Pphi15-msfGFP reporter construct. All samples were induced with 5 mM Rha for 12 h. Bars and error bars represent the mean 5(6)-FAM/OD₆₀₀ value and standard error of four biological replicates, respectively. Samples not connected by the same letters are significantly different (Tukey HSD, α=0.05).
C. Assessment of the inhibitory strength of the phi15 lysozyme (G3RQ) mutant in P. aeruginosa PAO1. The wild-type phi15 lysozyme (WT) and phi15 lysozyme mutant (G3RQ) were introduced in P. aeruginosa, together with the phi15 RNAP and Pphi15-msfGFP reporter construct, and induced with 5 mM Rha for 12 h. Bars and error bars represent the mean 5(6)-FAM/OD₆₀₀ value and standard error of four biological replicates, respectively. The significance level for a one-sided Student's t-test is indicated.

FIG. 8. The sequence downstream of T7-like phage promoters highly impact transcription efficiency.

For T7-like phages T7, phi15, PPPL-1, Pf-10 and 67PfluR64PP, their confirmed promoter was paired with different 5′UTRs: 1) the full 5′UTR of the corresponding phage's major capsid protein (MCP), 2) the standardized UTR BCD2, linked to the promoter by GGGCAG, 3) BCD2, linked to the promoter by the first two nucleotides of the corresponding phage's MCP and GCAG, 4) BCD2, linked to the promoter by the first thirteen nucleotides of the corresponding phage's MCP and GCAG. The combinations were cloned into pBGDes and introduced in P. putida KT2440 together with pSTDes3 carrying the corresponding phage RNAP. Bars represent the mean fluorescent intensity of four biological replicates after 6 h of induction with 0.3 3 mBz, expressed as equivalent 5(6)-FAM concentration and normalized for OD₆₀₀. Error bars represent the standard error.

FIG. 9. Phylogenetic tree of T7-like Pseudomonas phages and coliphage T7, based on the RNAP sequence.

Based on this tree, the phages can be subdivided into eleven different clades, indicated by different lines.

FIG. 10. Effect of phage RNAP expression on cell growth of P. putida KT2440 and P. aeruginosa PAO1.
A. All P. putida and P. aeruginosa strains RX (negative control), RA0 (T7), RB0 (phi15), RC0 (PPPL-1), RD0 (Pf-10) and RE0 (67PfluR64PP) were induced with 1 mM 3 mBz, after which the OD₆₀₀ was measured every 15 minutes for 12 hours. Markers and error bars represent the mean value and standard error of four biological replicates every 30 minutes.
B. Recognition of phage promoter by host RNAP of P. putida and P. aeruginosa. All P. putida and P. aeruginosa strains pX (negative control), pA0 (T7), pB0 (phi15), pC0 (PPPL-1), pD0 (Pf-10) and pE0 (67PfluR64PP) were monitored for 12 h, with OD₆₀₀ and msfGFP measurements every 15 minutes. Markers and error bars represent the mean value and standard error of four biological replicates every 30 minutes.
C and D. T7-like phage RNAPs generate high msfGFP expression levels from their putative phage promoter in P. putida KT2440 (C.) and P. aeruginosa PAO1 (D.). P. putida and P. aeruginosa strains pXRX (negative control), pA0RA0 (T7), pB0RB0 (phi15), pC0RC0 (PPPL-1), pD0RD0 (Pf-10) and pE0RE0 (67PfluR64PP) were induced with 0.3 mM 3 mBz, after which the OD₆₀₀ and fluorescent intensity was measured every 15 minutes for 12 hours. The fluorescent intensity was normalized for OD and expressed as equivalent 5(6)-FAM concentration (nM). Markers and error bars represent the mean value and standard error of four biological replicates every 30 minutes.

FIG. 11. Distinct promoter regions of T7-like promoters.
A. The T7 promoter consist of three main regions, 1) an N-terminal AT-rich recognition loop, 2) a specificity loop and 3) an unwinding region.
B. Transcription start site (TSS) determination of phage terminators with “-capping RACE. The template switching oligonucleotide (TSO) is directly linked to the 5′ terminus of the mRNA transcript and indicates the TSS of the phage promoter.
C. Clustal-omega alignment of the validated promoter and 5′ UTR of the phages' major capsid protein (MCP 5′UTR).

FIG. 12. Toxicity assay of phage lysozymes in P. putida KT2440 and P. aeruginosa PAO1.
A. P. putida and P. aeruginosa strains LX0 (negative control), LA0 (T7), LB0 (phi15), LC0 (PPPL-1), LD0 (Pf-10) and LE0 (67PfluR64PP) were induced with 10 mM Rha at OD₆₀₀0.3, after which cell growth was monitored every half hour for 12 h. Datapoints represent the mean OD₆₀₀ value of four biological replicates. Error bars represent the standard error.
B. Fluorescence assay to analyze the inhibitory effect of the phage lysozyme on its corresponding phage RNAP. P. putida strains pXRXLX (negative control), pA0RA0LA0 (T7), pB0RB0LB0 (phi15), pC0RC0LC0 (PPPL-1), pD0RD0LD0 (Pf-10) and pE0RE0LE0 (67PfluR64PP) were induced with 4 mM Rha, after which the fluorescence intensity and cell growth is monitored every half hour for 12 h. Datapoints represent the mean 5(6)-FAM/OD₆₀₀ value of four biological replicates. Error bars represent the standard error.
C. Fluorescence assay to analyze the inhibitory effect of the phage lysozyme on its corresponding phage RNAP. P. aeruginosa strains pXRXLX (negative control), pA0RA0LA0 (T7), pB0RB0LB0 (phi15), pC0RC0LC0 (PPPL-1), pD0RD0LD0 (Pf-10) and pE0RE0LE0 (67PfluR64PP) were induced with 5 mM Rha, after which the fluorescence intensity and cell growth is monitored every half hour for 12 h. Datapoints represent the mean 5(6)-FAM/OD₆₀₀ value of four biological replicates. Error bars represent the standard error.

FIG. 13. Preliminary assay to determine the ideal rhamnose concentration for induction of phage lysozymes.

P. putida strains pA0RA0 (T7 (−lys)) and pA0RA0LA0 (T7 (+lys)) were induced with 0-20 mM Rha for 12 h. Bars and error bars represent the mean 5(6)-FAM/OD₆₀₀ value and standard error of three technical replicates.

FIG. 14. Fluorescence assay to analyze the inhibitory effect of the phi15 lysozyme on its corresponding phage RNAP under different inducer concentrations.

P. putida strains pXRXLX (negative control) and pB0RB0LB0 (phi15) were induced with 0-100 mM Rha for 12 h. Bars and error bars represent the mean 5(6)-FAM/OD₆₀₀ value and standard error of four biological replicates.

FIG. 15. Fluorescence intensity assay to analyze the inhibitory effect of different phage lysozyme on phi15 RNAP.
A. P. putida strains pXRXLX (negative control), pB0RB0LA0 (T7), pB0RB0LB0 (phi15), pB0RB0LC0 (PPPL-1), pB0RB0LD0 (Pf-10) and pB0RB0LE0 (67PfluR64PP) were induced with 4 mM Rha at OD₆₀₀0.3, after which the fluorescence intensity and cell growth is monitored every half hour for 12 h. Datapoints represent the mean 5(6)-FAM/OD₆₀₀ value of four biological replicates. Error bars represent the standard error.
B. Fluorescence assay to assess the inhibitory strength of different phi15 lysozyme mutants on phi15 RNAP. P. putida strains pB0RB0LB0 (phi15 (WT)), pB0RB0LD0 (Pf-10 (WT)), pB0RB0LB1 (phi15 (AA1-9>Pf10 (AA1-10)), pB0RB0LB2 (phi15 (G3R)), pB0RB0LB3 (phi15 (G3Q)), pB0RB0LB4 (phi15 (G3RQ)), pB0RB0LB5 (phi15 (K5Q)) and pB0RB0LB6 (phi15 (K7N,E8K)) were induced with 5 mM Rha at OD₆₀₀0.3, after which the fluorescence intensity and cell growth is monitored every half hour for 12 h. Datapoints represent the mean 5(6)-FAM/OD₆₀₀ value of four biological replicates. Error bars represent the standard error.
C. Fluorescence assay to assess the inhibitory strength of phi15 lysozyme mutant G3RQ on phi15 RNAP. P. aeruginosa strains pB0RB0LB0 (phi15 (WT)) and pB0RB0LB4 (phi15 (G3RQ)) were induced with 5 mM Rha at OD₆₀₀0.3, after which the fluorescence intensity and cell growth is monitored every half hour for 12 h. Datapoints represent the mean 5(6)-FAM/OD₆₀₀ value of four biological replicates. Error bars represent the standard error.

FIG. 16. Initial validation set-up of the phi15 transcriptional system.
A. The transcriptional machinery of phage phi15 was introduced and validated in P. putida and P. aeruginosa, using a genomically integrated reporter construct with the phi15 promoter (Pphi15). The phi15 RNAP will recognize Pphi15 upon expression from the XyIS/Pm system and generate high expression levels. In uninduced conditions, high stringency can be obtained by inhibiting the basal levels of phi15 RNAP with phi15 lysozyme (G3RQ), which is expressed from the RhaRS/P_{rhaBAD} system.
B. The optimized phi15-based expression system for P. putida. Optimizations to the original set-up of A. include 1) genomic integration of the phi15 RNAP expression construct with a weak RBS, 2) a fully optimized expression vector for high-yield, reliable expression of a gene of interest, 3) genomic integration of the phi15 lysozyme (G3RQ) expression construct with a weak constitutive promoter and 4) an optional growth-decoupling module with phi15 gp16 for inhibition of the host RNAP. RNAP: RNA polymerase, lys: lysozyme, RBS: ribosomal binding site.

FIG. 17. Phi15 RNAP amplifies the output from traditional expression systems.

The performance of five different expression systems (XyIS/Pm, AraC/ParaBAD, RhaRS/PrhaBAD, LacI/PlacUV5 and a—responsive riboswitch) was evaluated while expressing either msfgfp (−RNAP) or phi15rnap (+RNAP) in LB medium and M9 minimal medium with different inducer concentrations. When phi15rnap was cloned under control of the expression system, an additional reporter construct with Pphi15 and msfgfp was introduced in the cell. All samples were grown to OD₆₀₀0.1 before induction with the relevant inducer concentration. Induced samples were incubated for 12 h before endpoint measurements of OD₆₀₀ and fluorescence were performed. Bars and error bars indicated the mean normalized fluorescence (5(6)-FAM/OD₆₀₀) and standard error of four biological replicates. Fold induction (FI) represents the ratio of the maximal and minimum msfGFP output.

FIG. 18. Genomic integration of XyIS/Pm::phi15rnap in P. putida KT2440 and SEM11 does not impact cell growth.
A. The phi15 RNAP integration cassette is integrated in three different loci: PP13, PP5042 and PP5322.
B. The genomic locus where phi15 RNAP is integrated has a significant influence on the fluorescent intensity, but does not impact cell growth. P. putida KT2440 and SEM11 strains without phi15 RNAP (NC), vector-borne expression of phi15 RNAP (pSTDesX), or phi15 RNAP integrated in one of three different genomic loci (PP13, PP5322 and PP5042) were induced with 0.3 mM 3 mBz at OD₆₀₀0.1, after which the fluorescence intensity and cell growth is monitored every half hour for 12 h. Datapoints represent the mean OD₆₀₀ and 5(6)-FAM/OD₆₀₀ values after 12 h of induction of four biological replicates. Fold induction (FI) represents the ratio of 5(6)-FAM/OD₆₀₀ levels with and without induction, with correction for the negative control (NC). Error bars represent the standard error. Samples not connected by same letters are significantly different (Tukey HSD, α=0.05).
C. A weaker RBS and startcodon to drive phi15 RNAP expression improves the dynamic range of the phi15 expression system. P. putida SEM11 strains without phi15 RNAP (NC) or phi15 RNAP integrated in PP13 with three different RBSs (original RBS, RBS-C and RBS-D) were induced with 0.3 mM 3 mBz at OD₆₀₀0.1, after which the fluorescence intensity and cell growth is monitored every half hour for 12 h. Datapoints represent the mean OD₆₀₀ and 5(6)-FAM/OD₆₀₀ values after 12 h of induction of four biological replicates.

FIG. 19. Genomic integration of P_{14c}-BCD22-phi15lys (G3RQ) in P. putida SEM11 improves the dynamic range of the phi15 expression system.
A. The phi15 lysozyme integration cassette is integrated in two different loci: PP4305 and PP5388.
B. The phi15 lysozyme (G3RQ) reduces basal expression and improves the dynamic range of the phi15 expression system. P. putida SEM11 without phi15 RNAP (NC), with the phi15 RNAP in PP13 with either RBS-C(RBS-C-lys) or RBS-D (RBS-D-lys) and with the phi15 lysozyme (G3RQ) in locus PP4305 or PP5388 (RBS-D+lys (PP4,305) and RBS-D+lys (PP5,388) were induced with 0.3 mM 3 mBz at OD₆₀₀0.1, after which the fluorescence intensity and cell growth is monitored every half hour for 12 h. Datapoints represent the mean 5(6)-FAM/OD₆₀₀ values after 12 h of induction of four biological replicates. Fold induction (FI) represents the ratio of 5(6)-FAM/OD₆₀₀ levels with and without induction, with correction for the negative control (NC). Error bars represent the standard error. Samples not connected by same letters are significantly different (Tukey HSD, α=0.05).

FIG. 20. Optimized expression vector pPUT delivers high expression levels of the gene-of-interest.
A. Linear vector map of pPUT, with R6K and oriT origins (R6K and oriT), kanamycin and gentamycin resistance markers (kanR and gmR), transposase Tn7 recognition sites (Tn7L and Tn7R), transcriptional terminators rrnB T1, LUZ7 T50, phi15 T5, phi15 T1 and lambda TO, phi15 promoter region phi15-BCD05 and a removable fluorescence cassette for cloning (p14g-BCD2-msfgfp) flanked with BsaI recognition sites.
B. The pPUT vector series is compatible with SEVAtile shuffling (ST) and Golden Standard (GS) assembly (level 1). All possible assemblies of transcription units with SEVAtile shuffling and Golden Standard assembly with pPUT-ST, pPUT-GS, pPUT-GSN, pPUT-GS2C and pPUT-GS2N2C are illustrated.
C. All optimization steps to create vector pPUT. P. putida SEM11 P15 strains with pBGDes.Pₚₕᵢ₁₅-msfgfp, pBGDes.LUZ7T50-Pₚₕᵢ₁₅-msfgfp, pBGDes.P_{phi15-BCD05}-msfgfp, pBGDes. Pphi15-msfgfp-phi15T5-phi15T1 and pPUT.msfGFP (top to bottom) were induced with 0.3 mM 3 mBz at OD₆₀₀0.1, after which the fluorescence intensity and cell growth is monitored every half hour for 12 h. Datapoints represent the mean 5(6)-FAM/OD₆₀₀ values after 12 h of induction of four biological replicates. Fold induction (FI) represents the ratio of 5(6)-FAM/OD₆₀₀ levels with and without induction. Error bars represent the standard error. Samples not connected by same letters are significantly different (Tukey HSD, α=0.05).

FIG. 21. Phi15 gp16 enables growth-decoupled production of msfGFP by inhibiting the host RNA polymerase.

Fluorescence intensity assay to analyze the growth-decoupling effect of different phage ORFs on P. putida. P. putida strains pB0RB0GD0 (negative control), pB0RB0GD1 (LUZ24 gp9 (Igy)), pB0RB0GD2 (LUZ19 gp28 (Rac)) and pB0RB0GD3 (phi15 gp16) were induced with 10 mM Rha and 0.3 mM 3 mBz at OD 600 0.1, after which the fluorescence intensity and cell growth is monitored every half hour for 12 h. Datapoints represent the mean OD₆₀₀ values (top) and mean 5(6)-FAM/OD₆₀₀ values (bottom) of four biological replicates. Error bars represent the standard error.

FIG. 22. Genomic integration of P_{14c}-BCD22-phi15lys (G3RQ) in P. putida SEM11 improves the dynamic range of the phi15 expression system.

The phi15 lysozyme (G3RQ) reduces basal expression and improves the dynamic range of the phi15 expression system. P. putida SEM11 without phi15 RNAP (NC), with the phi15 RNAP in PP13 with either RBS-C(RBS-C-lys) or RBS-D (RBS-D−lys) and with the phi15 lysozyme (G3RQ) in locus PP4305 or PP5388 (RBS-D+lys (PP4,305) and RBS-D+lys (PP5,388) were induced with 0.3 mM 3 mBz at OD 600 0.1, after which the fluorescence intensity and cell growth is monitored every half hour for 12 h. Datapoints and error bars represent the mean OD₆₀₀ and 5(6)-FAM/OD₆₀₀ values and standard error of four biological replicates.

FIG. 23. Selection of the RBS for expression of phi15 lysozyme G3RQ. P. putida KT2440 PP13::phi15rnap (RBS) without lysozyme (−lys) and with pSTDesR.phi15lysozyme (G3RQ) with different RBSs (BCD2, BCD13 or BCD22). All strains were induced with 0.3 mM 3 mBz at OD₆₀₀0.1, after which the fluorescence intensity and cell growth is monitored every half hour for 12 h. Datapoints and error bars represent the mean OD₆₀₀ and 5(6)-FAM/OD₆₀₀ values and standard error of four biological replicates after 12 h of induction.

FIG. 24. Optimized expression vector pPUT delivers high expression levels of the gene-of-interest.

All optimization steps to create vector pPUT. P. putida SEM11 P15 strains with pBGDes.Pₚₕᵢ₁₅-msfgfp, pBGDes.LUZ7T50-Pₚₕᵢ₁₅-msfgfp, pBGDes.P_{phi15-BCD05}-msfgfp, pBGDes. Pphi15-msfgfp-phi15T5-phi15T1 and pPUT.msfGFP (top to bottom) were induced with 0.3 mM 3 mBz at OD₆₀₀0.1, after which the fluorescence intensity and cell growth is monitored every half hour for 12 h. Datapoints and error bars represent the mean and standard error of 5(6)-FAM/OD₆₀₀ values of four biological replicates. Fold induction (FI) represents the ratio of 5(6)-FAM/OD₆₀₀ levels with and without induction

FIG. 25. The copy number of the reporter construct has a large impact on cell growth and fluorescence intensity.

Fluorescence intensity assay to analyze the effect of plasmid copy number of P_{phi15,MCP}-msfGFP on cell growth and fluorescence intensity in P. putida. P. putida strains pB0RB0 (pSTDesX), G1pB0 (PP13—pBGDes), G1pB0v2 (PP13-pSEVA621), G2pB0 (PP5,322), G2pB0v2 (PP5,322-pSEVA621), G3pB0 (PP5,042), G3pB0v2 (PP5,042-pSEVA621), G3pB0v3 (PP5,042-pSEVA631) and G3pB0v5 (PP5,042-pSEVA651) were induced with 0.3 mM 3 mBz at OD₆₀₀0.1, after which the fluorescence intensity and cell growth is monitored every half hour for 12 h. Datapoints represent the mean OD₆₀₀ values (top), mean 5(6)-FAM values (middle) and mean 5(6)-FAM/OD₆₀₀ values (bottom) of four biological replicates. Error bars represent the standard error.

FIG. 26. Fluorescence intensity assay to assess the basal expression level of reporter constructs with an upstream terminator in P. putida.
A. P. putida KT2440 strains with a Pphi15-msfgfp reporter construct with upstream terminator LUZ7 T7, LUZ7 T32, LUZ7 T47, LUZ7 T50, LUZ7 T50i, LUZ7 T60, LUZ100 T6, LUZ100 T16 or LUZ100 T19 were monitored for fluorescence intensity and cell growth for 12 h. Bars and error bars represent the mean msfGFP/OD 600 values and standard error of four biological replicates after 12 h. Samples not connected by the same letter are significantly different (Tukey HSD, α=0.05).
B. Fluorescence intensity assay to assess the potential influence of an upstream terminator on expression of the reporter construct in P. putida. P. putida KT2440 strains with pSTDesX.phi15rnap and a Pphi15-msfgfp reporter construct with upstream terminator LUZ7 T7, LUZ7 T32, LUZ7 T47, LUZ7 T50, LUZ7 T50i, LUZ7 T60, LUZ100 T6, LUZ100 T16 or LUZ100 T19 were induced with 0.3 mM 3 mBz at OD₆₀₀0.1, after which the fluorescence intensity and cell growth is measured for 12 h of induction. Bars and error bars represent the mean msfGFP/OD₆₀₀ values and standard error of four biological replicates after 12 h. Samples not connected by the same letter are significantly different (Tukey HSD, α=0.05)

FIG. 27: Conceptual comparison of phi15 MCP 5′UTR, BCD2 and phi15-based BCDs.
A. schematic overview of phi15 MCP 5′UTR (top), BCD2 (middle) and phi15-based BCDs
B. Leader peptide sequence and amino acid (AA) sequence of the different phi15-based BCDs.
C. Fluorescence assay to assess the translational strength of different phi15-based BCDs. For the msfGFP reporter (left), P. putida strains pB0RB0 (phi15MCP), pB2RB0 (BCD2), pB4RB0 (phi15BCD01), pB5RB0 (phi15BCD02), pB6RB0 (phi15BCD03), pB7RB0 (phi15BCD04) and pB8RB0 (phi15BCD05) were induced with 0.3 mM 3 mBz at OD₆₀₀0.3, after which the fluorescence intensity and cell growth is monitored every half hour for 12 h. A similar assay was performed for the mScarlet-I reporter (right), with P. putida strains pB9RB0 (phi15MCP), pB10RB0 (BCD2), pB11RB0 (phi15BCD01), pB12RB0 (phi15BCD02), pB13RB0 (phi15BCD03), pB14RB0 (phi15BCD04) and pB15RB0 (phi15BCD05). Bars represent the mean 5(6)-FAM/OD₆₀₀ value of four biological replicates at 12 h post induction. Error bars represent the standard error.

FIG. 28. terminator trap to determine the terminator efficiency of phage terminators for the phi15 RNAP.
A. General lay-out of the terminator trap.
B. Calculation of the terminator efficiency. Empty refers to a terminator trap without terminator.
C. Fluorescence intensity assay to assess the terminator efficiency of phage terminators for phi15 RNAP in P. putida. P. putida KT2440 strains with pSTDesX.phi15rnap and a Pphi15-msfgfp-terminator-BCD1-mCherry reporter construct with terminator phi15 T1, phi15 T3, phi15 T5, phi15 T5s, LUZ7 T50, LUZ7 T50i, LUZ7 T61, LUZ100 T6, LUZ100 T19 or T7 T7 were induced with 0.3 mM 3 mBz at OD₆₀₀0.1, after which the fluorescence intensity and cell growth is measured after 16 h of induction. Bars represent the mean msfGFP/OD 600 values (left), mean mCherry/OD₆₀₀ values (middle) and mean terminator efficiency values (right) of four biological replicates. Error bars represent the standard error.
D. Assay identical to C., with terminator phi15 T1, phi15 T5 or terminator pair phi15 T1+T5 or phi15 T5+T1.

Despite the widespread applications of the T7 transcriptional machinery in E. coli, its implementation in the Pseudomonas species has remained restricted due to the troublesome cytotoxicity of the T7 RNAP in this genus, as observed in this study as well. In the present invention, we mined four viral RNAPs from T7-related Pseudomonas phages, phi15, PPPL-1, Pf-10, and 67PfluR64PP, of which none impacted the fitness of SynBio host P. putida. In addition, these RNAPs displayed a broad range of transcriptional activity, a high level of orthogonality towards each other, and orthogonality to the host RNAP. This is in contrast to minor T7 promoter recognition that was observed for the host machinery. Two of the phage RNAPS, phi15 and PPPL-1 RNAP, also showed significant activity in P. aeruginosa, while no transcriptional activity was observed from the Pf-10 and 67PfluR64PP RNAPs in this host. The reason for this inactivity remains unclear but could potentially be due to improper expression of the RNAP genes, as they were not codon-optimized to the host.

Although the T7-like RNAPs did not display the same extremely high transcriptional activity as the T7 RNAP control, this is not considered a disadvantage. Their non-toxicity, full host orthogonality, and more balanced activity allow easier cloning and flexibility toward different experimental setups compared to their T7 counterpart [Kushwaha et al. (2014) Nat. Commun. 6, 7832; Liang et al. (2018) ACS Synth. Biol. 7, 1424-1435]. Furthermore, this mini orthogonal RNAP library enables a considered selection of an RNAP for a specific application, in which phi15 and PPPL-1 RNAP are more suited towards applications that require high-yield of the gene of interest, while Pf-10 and 67PluR64PP RNAP enable more tightly-regulated and balanced expression of toxic intermediates or end products. For example, high amounts of the fluorinase enzyme are required for in vivo biofluorination with P. putida, whereas overexpression of the glycolate oxidase enzyme in P. putida allows efficient ethylene glycol conversion into polyhydroxyalkanoates. On the other hand, tight expression control is preferred for endolysin expression for the controlled cell lysis of putida [Martínez et al. (2011) Microb. Biotechnol. 4, 533-547] and the study of toxic phage proteins in P. aeruginosa [Ceyssens et al. (2020) Viruses 12, 976].

Furthermore, the orthogonal RNAP library allows the creation of various AND gates, OR gates, and resource allocators. Due to the modularity of the T7-like RNAPs, these enzymes can be split into an enzymatic module and a promoter-recognition module. An AND gate is created by placing the two modules under the control of different inducible promoters and the desired output under the phage promoter, which only yields the output when both inducers are present. In addition, the enzymatic module of one RNAP can be paired with the promoter-recognition module of other phage RNAPs, thus enabling the creation of multiple AND gates in parallel, which all rely on the same core module. This concept was coined as a resource allocator, as the total amount of output solely depends on the core module and does not increase and overburden the cell when multiple promoter-recognition modules are expressed simultaneously. Thirdly, the T7 and phi15 RNAPs can be assembled into an AND gate in combination with the T7 promoter. When either the T7 or the phi15 RNAP are expressed, the desired output will be produced—though in lower amounts by the phi15 RNAP.

The remarkable transcriptional activity of viral RNAPs often leads to high levels of leaky expression of the gene of interest under uninduced conditions. This was addressed by introducing the corresponding phage lysozymes in the expression hosts, mirroring the proven strategy of the T7 system. While the phage RNAPS showed high specificity towards their native phage promoter, the phage lysozymes proved to be much more promiscuous. Indeed, the Pf-10 lysozyme reduced leakiness from the phi15 RNAP by 84%, whereas the native phi15 lysozyme only showed a 30% reduction in leaky msfGFP expression. These results inspired a directed mutation analysis of the phi15 lysozyme for improved RNAP inhibition, leading to the engineering of the high-performant, non-toxic phi15 lysozyme (G3RQ) mutant. In addition, the performance of the 67PfluR64PP and PPPL-1 lysozymes could be improved to reduce the basal expression from their corresponding RNAPs. Furthermore, the toxicity observed upon overexpression of the phage lysozymes can be alleviated by knocking out the amidase activity of these enzymes, as this activity is the likely source of the observed toxicity. Overall, the present invention provides a set of non-toxic, orthogonal viral RNAPs with well-defined promoter sequences and lysozyme-based RNAP repressors for the Pseudomonas species to expand the SynBio toolbox of this genus and allow the design of a plethora of synthetic genetic circuitry. Further improvements include increased genomic stability with genomic integration of the phage RNAP and an optimized and standardized reporter construct with reliable promoter variants and potent transcriptional terminators.

### T7-Like Pseudomonas Phage Genomes Encode Putative RNA Polymerases, Lysozymes, and Phage-Specific Promoters

Previous work has shown that the T7 RNAP causes significant growth deficits in Pseudomonas cultures upon expression. To reduce this cytotoxicity for Pseudomonas, one could employ two strategies: (1) optimize the T7 RNAP with directed evolution for Pseudomonas or (2) identify novel and optimized phage RNAPs from Pseudomonas phages. In the present invention, the latter option was chosen and focused on exploring the existing diversity of RNAPs among Pseudomonas phages for reduced cytotoxicity and increased transcriptional activity. While all Autographiviridae phages typically encode a viral RNAP, our analysis focused on T7-like phages, as they generally encode a small, single-subunit RNAP with clearly delineated promoter recognition sequences. To date, 36 T7-like Pseudomonas phages have been isolated and fully sequenced (FIG. 9). Based on the sequence alignment of these phage RNAPs, they can be subdivided into eleven distinct clades. We chose four phages from different clades (PPPL-1, Pf-10, 67PfluR64PP, and phi15) and analysed their annotated RNAP, lysozyme, and predicted-consensus promoter with several toxicity- and fluorescence-based assays. The genomic organization of phages T7, phi15, PPPL-1, Pf-10, and 67PfluR64PP and their promoter region preceding the major capsid protein (MCP) is illustrated in FIG. 2.

Screening Non-Toxic Phage RNA Polymerases and their Transcriptional Activity

First, the four phage RNAPs were screened for low cytotoxicity in P. putida and P. aeruginosa, compared to the T7 reference model. As the Pseudomonas phages co-evolved with their host, their early-expressed RNAP would have prime efficient production of viral particles and not trigger the host's toxicity. To confirm this, the RNAP from the selected Pseudomonas phages, Pf-10, phi15, PPPL-1, and 67PfluR64PP, were cloned into pSTDesX, introduced in either P. putida or P. aeruginosa and induced with 1 mM 3 mBz from the XyIS/Pm expression system. This expression system is considered the golden standard for P. putida and has successfully driven T7 RNAP expression in previous research to circumvent LacI-related regulatory issues in Pseudomonas [Herrero et al. cited above, Beentjes et al. cited above]. As anticipated, the T7-like RNAPs did not significantly reduce the final OD₆₀₀ of the host after 12 h of induction, with the exception of some limited growth reduction induced by the expression of the 67PfluR64PP RNAP in P. aeruginosa (Tukey HSD, p<0.001) (FIG. 3A). By contrast, the T7 RNAP caused a significant growth stop and growth retardation in both species, as anticipated (Tukey HSD, p<0.0001) (FIGS. 3A and 10A).

To include the T7 RNAP as a positive control in further assays, its inducer concentration is reduced from 1 mM to 0.3 mM 3 mBz in subsequent experiments to limit the toxic effect. Next, the transcriptional activity of the RNAPs was assessed indirectly by measuring the level of the msfGFP (monomeric superfolder green fluorescent protein) fluorescence from a phage promoter-msfGFP reporter construct. The predicted phage promoters and 5′ untranslated regions (UTRs) from the phages' major capsid protein (MCP) are shown in FIG. 2B and were cloned upstream of the msfGFP gene in the pBGDes. The reporter construct in the pBGDes was genomically integrated as a single copy in the host's Tn7 attB site to limit noise from the copy number differences. These reporter constructs were first tested individually in the host in the absence of the phage RNAPs to confirm whether the phage promoters are not recognized by the host RNAP, which would be indicated by a lack of msfGFP expression (FIG. 3B). In both P. putida and P. aeruginosa, no significant gene expression was observed from any of the phage promoters by the host RNAP, except for the T7 promoter. This result indicate that the T7 promoter can be recognized by the host RNAP (albeit very weakly) and is therefore not fully orthogonal to the host RNAP in Pseudomonas.

Next, all of the phage RNAPs were introduced in the corresponding reporter strains, and msfGFP expression was monitored for 12 h in the absence and presence of 0.3 mM of a 3 mBz inducer. All of the tested phage RNAPs displayed significant transcriptional activity in P. putida after 12 h of induction (pairwise Wilcoxon, p<0.001), whereas only T7, phi15, and PPPL-1 RNAP produced significant msfGFPs in P. aeruginosa (pairwise Student's t-test, p<0.05 (T7, phi15) and p>0.05 (PPPL-1); individual Student's t-tests for PPPL-1 vs. NC, p<0.05) (FIG. 3C). The reason for the apparent inactivity of Pf-10 and 67PfluR64PP RNAP in P. aeruginosa is unclear but could be due to improper expression of these RNAPs in this species, as the RNAP genes were not codon-optimized to the respective hosts. In the current setup, the T7 RNAP generated the highest msfGFP production per cell of 1185 nM and 1972 nM 5(6)-FAM/OD₆₀₀ in P. putida and P. aeruginosa after 12 h of induction, followed by 543 nM and 512 nM 5(6)-FAM/OD 600 for phi15 RNAP, respectively. In P. putida, the phage RNAPs of PPPL-1, Pf-10, and 67PfluR64PP showed reduced expression levels. Taken together, these less-active enzymes provide an RNAP library together with T7 and phi15 RNAP, covering a broad range of transcriptional expression. In this regard, it is noted that while the transcriptional activity of T7 RNAP remained highest in this experiment, its cytotoxicity and the impact this brings for cloning, handling, and expressing the T7 RNAP in Pseudomonas is to be considered. As such, a non-toxic RNAP with more average expression levels like the phi15 RNAP is preferable for many applications.

Looking back at the MCP promoter region of the phages (FIG. 2B), extensive conservation of the promoter motif can be observed, which could indicate the cross-recognition of the promoters by the phage RNAPs. To test if any cross-recognition occurred, all 25 combinations of the T7, phi15, PPPL-1, Pf-10, and 67PfluR64PP RNAPs and promoters were set up in P. putida and induced with 0.3 mM 3 mBz. Surprisingly, nearly full orthogonality was observed between the phage RNAPs, as illustrated in FIG. 4. Except for the phage RNAP 67PfluR64PP, all of the RNAPs yielded high msfGFP levels from their native promoter, whereas the msfGFP levels originating from other phage promoters remained insignificant (pairwise Student's t-test, p>0.05). Only for the phi15 RNAP was minimal cross-recognition observed from the T7 promoter (86 5(6)-FAM/OD₆₀₀), while this effect was not observed for the T7 RNAP in combination with the phi15 promoter. As such, this set of phage RNAPs and promoters can not only be used to build synthetic AND gates and resource allocators, but the combination of the T7 and phi15 RNAPs with the T7 promoter can even allow the construction of an OR gate and many other setups.

### Phages Phi15, PPPL-1, Pf-10, and 67PfluR64PP Encode Short, 17 bp Promoters

The T7 promoter is a well-characterized 17 bp sequence with an N-terminal AT-rich recognition loop (−17-13), a specificity loop of 5 bp (−11-7), and an unwinding region (−4-1) (FIGS. 5 and 11). The predicted phage promoters of PPPL-1, Pf-10, 67PfluR64PP, and phi15 all show a high sequence similarity to the T7 promoter. As such, it is reasonable to assume that these promoters contain a similar structure. To validate the exact transcription start site (TSS) of the phage promoters in vivo, a 5′-capping-RACE experiment was performed using the P. putida KT2440 strains pA0RA0, pB0RB0, pC0RC0, pD0RD0, and pE0RE0. 5′capping-RACE (Rapid Amplification of cDNA Ends) allows the capture of full-length mRNA molecules. Subsequent sequencing of their 5′ termini precisely determines the TSSs.

The capping-RACE experiment confirmed the start sites of the predicted promoters of phi15, PPPL-1, Pf-10, and 67PfluR64PP. Overall, these T7-like promoters showed a canonical length of 17 bp (18 bp for P_{pf-10}) and two AT-rich regions flanking the presumed recognition loop (FIGS. 5 and 11). This validation allowed us to successfully pair the promoters with other 5′ UTRs, including BCD2, a standardized, highly-potent UTR with a bicistronic design commonly used in P. putida (FIG. 8).

These standardized bicistronic UTRs are of specific interest for the use of these phage promoters in synthetic circuitry. Indeed, BDC2 and other bicistronic designs have the advantage of circumventing the well-known problem of secondary structure formation between the RBS and the downstream gene of interest, potentially inhibiting proper translation. This allows the user to reliably reuse the expression construct in a standardized design for different genes of interest without the need for individually optimized 5′ UTRs for each construct.

### T7-Like Phage Lysozymes Inhibit Transcriptional Activity of Their Corresponding Phage RNAP

Due to the strong transcriptional activity of T7-like phage RNAPs, a limited production of the phage RNAP can rapidly lead to significant expression levels of the reporter gene in uninduced conditions. This observation can also be made for the uninduced P. putida samples from the previous assay, where all strains except the 67PfluR64PP RNAP produced msfGFP in significantly higher concentrations compared to the negative control (Tukey HSD, p<0.05) (FIG. 3C). The highest levels of leaky expression in P. putida are observed for phi15 (160 5(6)-FAM/OD₆₀₀) and T7 (120 5(6)-FAM/OD₆₀₀). This leaky msfGFP expression is likely caused by the low basal expression of the phage RNAP from the XyIS/Pm expression system. To limit this leaky expression, the corresponding phage lysozyme can be added to the system to inhibit the phage RNAP and therefore decrease the transcription of the reporter gene. As indicated in FIG. 2, the genomes of phi15, PPPL-1, Pf-10, and 67PfluR64PP all encode an early-expressed lysozyme with high similarity to the T7 lysozyme. This lysozyme could potentially inhibit the corresponding phage RNAP but could also exhibit cytotoxicity due to their intrinsic amidase activity, as shown by the overexpression of the T7 lysozyme. Therefore, all of the lysozymes were cloned into pSTDesR with the RhaRS/P_{rhaBAD} expression system, introduced into P. putida KT2440 and P. aeruginosa PAO1, induced with 10 mM Rha (rhamnose), and screened for the host's toxicity. Interestingly, all of the phage lysozymes significantly reduced the cell growth of P. aeruginosa (Tukey HSD, p<0.01), while in P. putida, no significant toxicity was observed from the expression of the phi15, PPPL-1, and Pf-lysozymes after induction (Tukey HSD, p>0.1) (FIG. 6A). This is in contrast to the moderate toxicity observed by the T7 and 67PfluR64PP lysozymes, respectively (Tukey HSD, p<0.01) (FIG. 6A). The rhamnose induction concentration in further assays will be reduced to 5 mM instead of 10 mM to limit the toxic effect but still allow sufficient lysozyme expression to inhibit the RNAP, as determined for the T7 system (FIG. 13).

Next, the inhibitory effect of the lysozymes on the phage RNAP was analyzed in P. putida and P. aeruginosa by introducing the phage lysozyme, RNAP, and phage promoter-msfgGFP reporter construct in the host and monitoring the msfGFP output after induction with 4 mM Rha. Upon the induction of lysozyme expression, all P. putida samples showed a significantly reduced msfGFP output compared to their uninduced counterparts (FIG. 6B), indicating that all of the lysozymes successfully inhibited their corresponding RNAP and reduced leaky expression. The largest reductions in the msfGFP were observed for the Pf-10 (−80%) and T7 (−79%) systems, followed by a medium reduction for the 67PfluR64PP (−51%) and PPPL-1 (−39%) systems. The phi15 lysozyme caused only a −30% reduction of the msfGFP output, therefore still displaying a significant level of leaky fluorescence of 77 nM 5(6)-FAM/OD₆₀₀. In P. aeruginosa, on the other hand, a slight reduction trend in the leaky expression was observed for T7, phi15, and PPPL-1 upon the lysozyme expression, but these reductions did not prove to be significant (one-sided Student's t-test, p>0.05). Therefore, the assay was repeated with 5 mM Rha instead of 4 mM to increase the lysozyme expression without causing significant growth retardation. As displayed in FIG. 6B, the msfGFP output of the induced P. aeruginosa strains now trended lower than the uninduced controls in all of the strains, but this was only statistically significant for the phi15 sample (one-sided Student's t-test, p<0.05). Overall, these results indicate that the predicted phage lysozymes can reduce basal msfGFP expression levels originating from the phage RNAPs and should be introduced to improve the stringency of the expression circuitry.

T7-Like Phage Lysozymes Efficiently Inhibit Phage RNAPs from Related T7-Like Phages

The viral RNAP from the phage phi15 resulted in high expression levels in P. putida and P. aeruginosa (FIG. 3) but also exhibited significant leakiness, even in the presence of the phi15 lysozyme (FIG. 6B). In an attempt to further reduce the leaky expression, the rhamnose concentration was increased up to 100 mM in P. putida, to no avail (FIG. 14). As shown for the T7 system, the lysozyme inhibitory action stems from its N-terminal tail, with which it binds to the phage RNAP and causes allosteric inhibition of this enzyme. Changes in the N-terminal tail sequence can, therefore, result in either weaker or stronger binding of the RNAP, which can, in turn, influence the inhibitory activity. The five studied phage lysozymes in the present invention show a range of inhibitory performances and encode distinct N-terminal regions (FIG. 15). Therefore, we paired the lysozymes of T7, phi15, PPPL-1, Pf-10, and 67PfluR64PP with the phi15 RNAP and Pphi15-msfGFP reporter construct to test whether these lysozymes also have different abilities to bind and inhibit the phi15 RNAP. The resulting P. putida strains were induced with 4 mM rhamnose to express the lysozyme, after which the msfGFP output was measured for 12 h (FIG. 7A). Despite a significant growth reduction caused by the 67PfluR64PP and Pf-10 lysozymes upon induction, similar to the previous assay, these lysozymes showed a remarked reduction in the leakiness caused by the phi15 RNAP. The 67PfluR64PP lysozyme reduced the leaky expression by 58%. Interestingly, the Pf-10 lysozyme even significantly outperformed the phi15 lysozyme with an 84% decrease in the msfGFP output (Tukey HSD, p<0.0001). To verify that this striking result was due to the unique N-terminal region of the Pf-10 lysozyme and not to any other amino acid differences between the sequences of the Pf-10 and phi15 lysozymes (or even due to the slight toxicity of the Pf-10 lysozyme), a phi15 lysozyme mutant was engineered in which the first nine N-terminal amino acids were substituted for the first ten amino acids of the Pf-10 lysozyme while maintaining the other 145 amino acids of the phi15 lysozyme. The resulting P. putida strain with the phi15 RNAP, reporter construct, and phi15 lysozyme (AA1-9>Pf-10 (AA1-10)) showed no reduction in cell growth and generated a fluorescent output that was nearly identical to the sample with the Pf-10 lysozyme when induced with 5 mM rhamnose (Tukey HSD, p>0.05) (FIG. 6B). This confirms the determining role of the N-terminal region on RNAP inhibition and strongly suggests that the N-terminal region of the Pf-10 lysozyme allows a stronger binding interaction with the phi15 RNAP than the phi15 lysozyme. To pinpoint the exact amino acids causing the increased inhibitory activity, five additional phi15 lysozyme mutants were created and introduced in P. putida with the phi15 RNAP and reporter construct: phi15 lysozyme (G3R), (G3Q), (G3RQ), (K5Q), and (K7N,E8K). Except for the G3Q mutant (p<0.01), none of the mutants had a significant impact on cell growth.

While the phi15 lysozyme mutants (G3Q), (K5Q), and (K7N,E8K) did not improve the inhibitory activity of the lysozyme (FIG. 7B), mutants (G3R) and (G3RQ) did decrease the leaky expression to 57 nM and 11 nM 5(6)-FAM/OD₆₀₀, respectively. This is a significant improvement compared to the 122 nM 5(6)-FAM/OD₆₀₀ observed for the wild-type phi15 lysozyme. Moreover, the results of the G3RQ mutant even outperformed those of the phi15 lysozyme (AA1-9>Pf-10 (AA1-10) (23 nM 5(6)-FAM/OD₆₀₀) (Tukey HSD, p<0.05). To confirm that this mutant functions in other Pseudomonads, the experimental setup was also analyzed in P. aeruginosa. In this host, the (G3RQ) mutant considerably reduced the leaky expression from the phi15 RNAP to 29 5(6)-FAM/OD₆₀₀ upon induction with 5 mM rhamnose, a remarkable 13-fold lower compared to the wild-type phi15 lysozyme (one-sided Student's t-test, p<0.01) (FIG. 7C).

The results also indicate that the third position in the amino acid sequence plays an important role in RNAP inhibition and that a charged amino acid (R,Q) is preferred over the small glycine residue in the phi15 lysozyme sequence to create a strong interaction with the phi15 RNAP. These results correspond to previous work where point mutations in the N-terminal region of the T7 lysozyme caused a lack of RNAP inhibition, thus showing that even single point mutations can significantly impact the lysozyme-RNAP interaction [Jeruzalmi et al. (1998) EMBO 35 J. 17, 4101-4113]. Lastly, it can be noted that there are also large differences in the msfGFP output between the strains in the uninduced condition (FIG. 6B), which could be attributed to the minor leaky expression of the lysozymes from the RhaRS/P_{rhaBAD} system. Overall, these results suggest that the other phage lysozymes could also be optimized for reduced toxicity and increased RNAP inhibition in a similar manner to enable tightly-controlled expression systems for SynBio applications.

### Flow Cytometry-Based Quantitative Assessment of the Phi15 Expression System

The phi15 RNAP and phi15 lysozyme (G3RQ) form a stringent expression system in P. putida and P. aeruginosa together with the phi15 promoter (FIG. 6B). To characterize this system on a single-cell level, a flow cytometry experiment was performed on the P. putida and P. aeruginosa wild-type strains, the strains with the phi15 RNAP, phi15 lysozyme, and reporter construct, and the strains with the phi15 RNAP, phi15 lysozyme (G3RQ), and reporter construct (Table 1).

TABLE 1
Flow cytometry data of the phi15 expression system in P. putida and P. aeruginosa.
Wild-type P. putida KT2440 and P. aeruginosa PAO1 strains (wild-types),
P. putida and P. aeruginosa with the phi15 RNAP, phi15 reporter construct,
and phi15 lysozyme (phi15), and P. putida and P. aeruginosa with the phi15 RNAP,
phi15 reporter construct, and phi15 lysozyme (G3RQ) mutant (phi15(G3RQ)) were
induced overnight with 5 mM Rha (+lys) or 0.3 mM 3mBz (+RNAP), after which
5000 cells were analysed with flow cytometry for FITC-A, as described in the
methods' section. Cells with a FITC-A level above 10⁴ are considered
induced, whereas cells below 10⁴ are uninduced. Column FITC-A
depicts the median FITC-A value of the entire cell population, and column
induced (%) depicts the percentage of cells of the entire population that have
a FITC-A value above 10⁴. Complete histograms of the corresponding
data are available in FIG. 17.

| | | P. putida | | P. putida | | P. putida | |
|---|---|---|---|---|---|---|---|
| | | Wild-Type | | phi15 | | phi15 (G3RQ) | |
| | | | Induced | | Induced | | Induced |
| | | FITC-A | (%) | FITC-A | (%) | FITC-A | (%) |
| −RNAP | +lys | 44 | 0.16 | 17,803 | 80.00 | 12,730 | 66.66 |
| −RNAP | −lys | 70 | 0.12 | 15,270 | 74.74 | 32,128 | 78.50 |
| +RNAP | −lys | 99 | 0.30 | 72,956 | 95.40 | 122,739 | 92.82 |
| Fold | | 2.25 | | 4.10 | | 9.64 | |
| induction* | | | | | | | |

| | | P. aeruginosa | | P. aeruginosa | | P. aeruginosa | |
|---|---|---|---|---|---|---|---|
| | | Wild-Type | | phi15 | | phi15 (G3RQ) | |
| | | | Induced | | Induced | | Induced |
| | | FITC-A | (%) | FITC-A | (%) | FITC-A | (%) |
| −RNAP | +lys | 647 | 3.22 | 33,429 | 77.52 | 527 | 5.94 |
| −RNAP | −lys | 692 | 6.18 | 7820 | 47.74 | 18,672 | 81.10 |
| +RNAP | −lys | 559 | 3.22 | 245,893 | 90.98 | 68,392 | 74.58 |
| Fold | | 0.86 | | 7.36 | | 129.78 | |
| induction* | | | | | | | |

*Fold induction is the ratio of FITC-A of the (−RNAP, +lys) sample and the (+RNAP, −lys) sample.

All of the P. putida and P. aeruginosa samples displayed single, homogenous populations, indicating that most of the cells responded to the presence of the inducers in a similar manner, with a limited occurrence of escapers. In addition, the wild-type controls showed very little response to the inducers in terms of the FITC-A (related to msfGFP expression). This allowed us to determine the threshold of the background FITC-A for P. putida and P. aeruginosa in this experiment, which was set at 10⁴. The results of the phi15 wild-type and phi15 (G3RQ) strains support the observations made in the previous spectrophotometric data (FIG. 6). Both in P. putida and P. aeruginosa, the presence of phi15 lys (G3RQ) reduced the median FITC-A and the size of the induced cell population, resulting in a significantly improved fold induction in comparison to the wild-type phi15 lysozyme (fold induction 9.64 vs. 4.10 for P. putida and 129.78 vs. 7.36 for P. aeruginosa, respectively) (Table 1). Remarkably, in P. aeruginosa, the phi15 lysozyme G3RQ reduced the FITC-A, even below the value observed for the wild-type strain (FITC-A 527 vs. 647, respectively). As such, these results confirm the superiority of the phi15 lysozyme (G3RQ) over its wild-type counterpart and illustrate the potential of the phi15 expression system as a tool in P. putida and P. aeruginosa.

Phage Promoters and their Native 5′UTR are Co-Evolved to Yield High Expression Levels

To validate the expression levels of the phage RNAPs and promoters in combination with BCD2, all phage promoters were connected to BCD2 by a GGGCAG linker and cloned together with msfGFP into pBGDes. The GGGCAG linker contains the GCAG position tag required for SEVAtile shuffling and a double G directly following the TSS of the promoter. This is known to be important for proper transcription initiation of the T7 promoter (FIG. 5). The resulting vectors were introduced together with the corresponding phage RNAP in P. putida KT2440. Unexpectedly, the replacement of the MCP 5′UTR by BCD2 resulted in a significant drop in msfGFP fluorescence output for all phage RNAPs except for Pphi15 (P<0.05) (FIG. 8). For Pf-10 and 67PfluR64PP, almost no fluorescent intensity was detected at all, indicating that the confirmed phage promoter is not sufficient for efficient transcription by these phage polymerases. A first hypothesis for this observation led us to the unwinding region of the phage promoters. As indicated in FIG. 4, the last four nucleotides of the promoters are predicted to play an important role in DNA unwinding. Interestingly, this region contains three A/T nucleotides for both the Pf-10 and 67PfluR64PP promoter, while the other promoters have four A/T nucleotides. Therefore, it is reasonable to assume that four consecutive A/T nucleotides are required for proper DNA unwinding and transcription initiation of these promoters.

To test this hypothesis, the GGGCAG linker from the previous construct was replaced by the two first nucleotides of the corresponding phage MCP 5′UTR followed by GCAG. In this way, all phage promoter-UTR constructs contain four consecutive A/T nucleotides, which will potentially increase the fluorescent output. This trend could indeed be observed for Pf-10 and 67PfluR64PP, but the msfGFP levels still remain about fourfold lower than the levels observed for the full MCP 5′UTR (FIG. 8). For T7, phi15 and PPPL-1, no significant difference in msfGFP output was observed between constructs containing the GGGCAG linker or NNGCAG linker. These results indicate that an intact unwinding region of four consecutive A/T nucleotides is important for T7-like promoters, but does not fully explain the significant difference in fluorescence intensity between the MCP 5′UTR and BCD2 for PPPL-1, Pf-10 and 67PfluR64PP (FIG. 5).

When analyzing the MCP 5′UTR further, it can be observed that all T7-like phage promoter regions in this paper contain a 13-nt stretch without any thymidine residue directly downstream of the TSS. The conservation of this T-less stretch in diverse T7-like phages could indicate that this region is important for efficient transcription by the phage RNAP. Therefore, the previous linkers are now extended with the thirteen first nucleotides of the corresponding phage's MCP 5′UTR to include the T-less stretch. The addition of the T-less stretch has a marked influence on the msfGFP expression levels of Pf-10 and 67PfluR64PP, while the effect on T7, phi15 and PPPL-1 is much less pronounced (FIG. 5). In case of 67PfluR64PP, the fluorescence intensity is sevenfold higher compared to the N13-GCAG-BCD2 UTR and almost twice as high as the MCP 5′UTR. However, for all other phages the MCP 5′UTR still outperforms all UTRs containing BCD2. These results once again highlight that the phage promoter and MCP 5′UTR have been evolutionarily optimized to generate high levels of transcription together and that splitting these two parts to used them separately in synthetic circuitry is not straightforward.

The Transcriptional Machinery of Phage Phi15 Shows Potential as a Phage-Based Expression System for P. putida

T7-like Pseudomonas phage phi15 encodes a single-subunit RNAP which generates high levels of transcription in Pseudomonas hosts from a short, 17 bp promoter (5′-TAAAAACCCACACAATA-3′) [SEQ ID NO: 5] with a negligible burden on cell fitness. Even with the high transcriptional activity of the phi15 RNAP, high stringency can be obtained by inhibition of the phi15 RNAP by phi15 lysozyme (G3RQ) in uninduced conditions.

The phi15 transcriptional system has been validated in P. putida KT2440 and P. aeruginosa POA1 in previous work, by combining a genomically-integrated Pphi15-msfgfp reporter construct with vector-borne expression of the RNAP and lysozyme (G3RQ) through the XyIS/Pm and RhaRS/P_{rhaBAD} systems, respectively (FIG. 16A). While the initial set-up already showed promising results of the transcriptional machinery of phi15 as an expression system for P. putida with fluorescence expression levels up to 543 nM 5(6)-FAM/OD₆₀₀, multiple points for improvement and optimization are apparent and will be explored in this work (FIG. 16B). These improvements include 1) more balanced expression of the phi15 RNAP from a genomically-integrated construct, 2) a fully optimized and standardized expression vector which is compatible with SEVAtile and Golden Standard cloning methods, 3) an integrated expression cassette to ensure high stringency, using the phi15 lysozyme G3RQ, and 4) an optional module for growth decoupling by host RNAP-inhibitor phi15 gp16.

### Balanced, Single-Copy Expression of Phi15 RNAP Improves the Dynamic Range of the System

The phi15 RNAP amplifies expression levels from traditional expression systems. The phi15 transcriptional system requires expression of phi15 RNAP by a host RNAP-driven expression system. The choice for a specific system is not trivial, as it will determine the homogeneity, stringency, dose-response and maximal expression levels of our final phi15-based expression system. As such, five different expression systems (XyIS/Pm, AraC/ParaBAD, RhaRS/PrhaBAD, LacI/PlacUV5 and an—responsive riboswitch) were selected to drive phi15 RNAP expression in P. putida KT2440 and analyzed in different conditions, namely rich LB medium and M9 minimal medium with five different inducer concentrations relevant to each system. The phi15 RNAP will then on its turn initiate msfGFP expression from the phi15 promoter. For comparison, control constructs where the expression systems directly drive msfGFP expression instead of phi15 RNAP were constructed and analyzed in parallel.

Overall, the results show that phi15 RNAP is successfully expressed from all five selected systems and increases the fluorescent output of the system compared to the corresponding msfGFP control (FIG. 17). This effect is most noticeable for the XyIS/Pm and LacI/PlacUV5 systems, where the samples with phi15 RNAP show a 7.9- and 11.1-fold higher fluorescence level in LB medium for the highest inducer concentrations and a 11.1 and 23-fold increase in fluorescence in M9 medium, respectively. Minor reductions of final cell densities were observed for the phi15 RNAP samples induced with the highest inducer concentrations (results not shown), which can be attributed to the high production levels of msfGFP causing cell burden. While the results show the highest fluorescence levels when phi15 RNAP is expressed with the LacI/PlacUV5 system, this system also lacks stringency and displays limited tunability. This is in contrast to the AraC/ParaBAD and RhaRS/P_{rhaBAD} systems, which are highly tuneable and stringent, but rely on expensive inducers. Based on these results, the XyIS/Pm is selected to express phi15 RNAP, as it requires the cheap 3-methylbenzoate (3 mBz) inducer, shows acceptable basal expression levels in uninduced conditions and a moderate level of tunability. These characteristics allow a broad-range of applications with the final system. For specific applications, the XyIS/Pm system can still be substituted for any other system if needed.

Stable Genomic Integration of Phi15 RNAP in Pseudomonas putida.

Due to the exceptional transcriptional activity of phi15 RNAP, small basal amounts of this enzyme immediately result in significant expression of the gene of interest even in uninduced conditions (FIG. 17). Previous work has shown that phi15 RNAP can be efficiently inhibited by phi15 lysozyme (G3RQ) to reduce leaky expression. However, also other approaches are explored to reduce basal phi15 RNAP expression from the XyIS/Pm system to improve the stringency and dynamic range of the system, while still maintaining high product yield upon induction. First, the copy number of the phi15 RNAP expression construct is reduced by switching from vector-born expression to stable, single-copy integration of the construct in P. putida KT2440 and P. putida SEM11, a genome-reduced production strain. Besides the reduction in copy number to reduce basal expression levels, genomic integration of genetic constructs is usually preferred over vector-based systems to reduce copy number-related noise and allow antibiotic-free cell culturing. Therefore, the expression cassette of pSTDesX.phi15RNAP was successfully integrated in three different loci, namely PP13, PP5322 and PP5042, to identify a genomic locus causing minimal cell burden upon integration and high expression levels (FIG. 18A).

To analyze the performance of these strains, a phi15 reporter construct was introduced into the hosts (Tn7attB::Pₚₕᵢ₁₅-msfgfp) and both the OD₆₀₀ and msfGFP output were monitored for 12 h in uninduced and induced conditions (0.3 mM 3 mBz). None of the genomic integrations caused a significant effect on cell fitness in comparison to wildtype P. putida KT2440 and SEM11 (Tukey HSD, P>0.05) (FIG. 18c), whereas a small but significant reduction in final OD₆₀₀ levels can be observed upon induction of our previous vector-based system with pSTDesX·phi15RNAP (Tukey HSD, P<0.05) (FIG. 18B).

Interestingly, all three genomic loci generate a different level of fluorescence intensity upon induction of the phi15 RNAP with 3 mBz. The highest levels can be observed for locus PP13, which is located closest to the origin of replication and yields 344 nM 5(6)-FAM/OD₆₀₀, an amount that is almost 50% higher than the pSTDesX-based system (234 nM 5(6)-FAM/OD₆₀₀) in P. putida KT2440 (Tukey HSD, P<0.05). It is surprising that a single-copy construct is outperforming the vector-based system in terms of expression levels, which could in part be explained by the reduced cell burden of phi15 RNAP overexpression. Locus PP5322 generates similar fluorescence levels as from the strain carrying pSTDesX.phi15 (226 nM 5(6)-FAM/OD₆₀₀), while the levels of locus PP5042 are slightly lower (184 nM 5(6)-FAM/OD₆₀₀). Depending on the application, different expression levels of the gene of interest could be desirable and one could prefer a different genomic integration locus. In the P. putida SEM11 host, similar trends can be observed, but this strain shows overall higher expression levels in comparison to his KT2440 counterpart. As such, in this work we will focus our efforts on further optimizing the P. putida SEM11 PP13::phi15rnap strain, as it generates the highest expression levels without metabolic burden on the host (FIG. 18B,C).

However, the initial aim of this experiment was improving the dynamic range and stringency of the system by reducing the copy number of the phi15 RNAP encoding construct, but no major improvements of the fold-induction levels or the basal expression levels were observed upon genomic integration of the phi15 RNAP. Therefore, another strategy was employed, where the RBS driving phi15 RNAP translation was replaced by two weaker variants (RBS-C and RBS-D), accompanied by the alternative GTG startcodon (FIG. 18C). We hypothesize that a weaker RBS and startcodon should reduce the basal expression levels of phi15 RNAP and, consequently, the Pphi15-msfgfp reporter construct, while also preventing premature saturation of the system with phi15 RNAP upon induction. Both RBS-C and RBS-D caused a significant reduction of basal msfGFP expression compared to the original RBS driving phi15 RNAP expression (Tukey HSD, P<0,0001), which improved the dynamic range of the system more than tenfold (FIG. 18C). More specifically, RBS-D shows a fold-induction level of 25.6, while still enabling induced expression levels of 300 5(6)-FAM/OD₆₀₀. As such, RBS-D is selected as a fixed element of the phi15-based expression system in the following optimization steps. RBS-C, on the other hand, shows the lowest leaky expression levels and highest fold-induction, but also highly impacts the msfGFP output after induction with 3 mBz.

### Introduction of Phi15 Lysozyme (G3RQ) Results in a Stringent Expression System

The genomic integration of phi15 RNAP in combination with a degenerate RBS and GTG startcodon already reduced leakiness of the system significantly and resulted in a higher dynamic range (FIG. 18C). To build on these results and reduce leakiness even further, phi15 lysozyme (G3RQ) is introduced in P. putida SEM11 PP13::phi15rnap (RBS-D). Initially, the lysozyme was under control of the RhaRS/P_{rhaBAD} system on a low-copy number vector. While this approach yielded satisfying results and high controllability of lysozyme expression, it does require the use of an additional inducer and an antibiotic. To avoid this in the optimized phi15-based expression system, constitutive expression is preferred from a stably-integrated marker-free construct. It is important to note that only very low expression levels of the lysozyme are desired, as we aim to only inhibit basal levels of phi15 RNAP in uninduced conditions. Therefore, P_{14c} and BCD22 were selected to drive lysozyme expression, as they are a weak promoter and RBS (FIG. 23). The resulting construct is genomically integrated in two different genomic loci, PP4503 and PP5388, which are known to yield minimal expression levels (FIG. 19A).

Upon integration of the phi15 lysozyme (G3RQ) in PP4305 or PP5388, the dynamic range of the system increased from 33 to 107- and 70-fold, respectively. Furthermore, the basal expression levels of these strains were indistinguishable from the negative control (Tukey HSD, P<0.05), showing that the phi15 lysozyme (G3RQ) significantly improves the tightness of the phi15 expression system. Due to the continuous expression of the phi15 lysozyme (G3RQ), also a reduction of expression levels in the induced state are observed, which are threefold less compared to the strain without lysozyme. As such, we will continue with both the strain without lysozyme (P. putida SEM11 PP0013::phi15rnap (RBS-D)) for non-toxic genes-of-interest and the strain with lysozyme (P. putida SEM11 PP13::phi15rnap (RBS-D) PP4305::phi15lysozyme (G3RQ)) for genes-of-interest or applications requiring tight regulatory control. These strains will further be called P. putida P15 and P. putida P15-L, respectively.

Optimized Expression Vector pPUT Enables High Production Levels of the Gene-of-Interest

A single-copy expression construct leads to healthy cells and maximal msfGFP output. In order to obtain high yields of the desired protein with minimal cell burden, it is important to integrate the phi15 promoter and gene-of-interest in the optimal genetic background, to ensure proper insulation from its surroundings and avoid the formation of secondary mRNA structures obstructing the RBS from binding to the ribosome. In the previous assays, the msfGFP reporter constructs were always genomically integrated in the host as a single copy. To determine the impact of the copy number on msfGFP production, the reporter construct Pₚₕᵢ₁₅-msfgfp is integrated in identical vectors with different origins of replication: pSEVA621 (RK2—low copy number), pSEVA631 (pBBR1—medium copy number), pSEVA641 (pR01600/ColE1—high copy number) and pSEVA651 (RSF1010—high copy number) and compared to the original single-copy construct (FIG. 24). All vectors were introduced in several P. putida backgrounds, with the phi15 RNAP present in different genomic loci.

Overall, the single-copy construct shows the least impact on cell growth, while still enabling very high absolute and normalized msfGFP expression levels. For higher copy number backbones, either no viable cells were obtained or cells showed severely reduced cell growth upon induction, resulting in low absolute msfGFP levels (FIG. 24). Based on these results, the original single-copy pBGDes vector, which integrates in the host's Tn7attB site, will be used as a backbone to create our final expression construct.

### Transcriptional Terminators Flanking the Expression Construct Ensure Genetic Insulation and Improve Expression Output.

Besides the vector copy number, efficient transcription termination and genetic insulation is also known to play a crucial role in circuit performance. Therefore, the expression construct is flanked with upstream and downstream terminators for proper insulation and termination of the phi15 RNAP. Terminator T50 from phage LUZ7 is placed upstream from the expression construct, as it is a strong, bidirectional terminator. It insulates the construct while causing minimal impact on the expression levels of the phi15 reporter construct in comparison to the control construct without additional terminators (FIG. 20B, FIG. 25). The terminator downstream of the reporter construct, on the other hand, is selected based on efficient termination of the phi15 RNAP and stabilization of the mRNA molecule, resulting in increased msfGFP output. A screen of ten potential terminators showed that placing phi15 terminators T5 and T1 in tandem resulted in a termination efficiency of 99.5% (FIG. 27). Furthermore, the terminator pair doubles the msfGFP output compared to the original terminatorless construct (823 vs. 385 nM 5(6)-FAM/OD₆₀₀, respectively) (Tukey HSD, p<0.001) (FIG. 20B). Phi15 BCDs. The efficiency of translation not only depends on the RBS sequence, but is also affected by secondary mRNA structure formation between the 5′ untranslated region (UTR) and the coding sequence. As such, identical RBS sequences can result in highly diverging translation efficiencies depending the coding gene. This problem can be avoided by incorporating the gene in a bicistronic rather than a monocistronic design. A bicistronic design consists of a standard leader peptide and the gene of interest. The initial RBS enables translational of the leader peptide, of which the sequence is optimized to avoid secondary structures.

Within the leader peptide, a second RBS is encoded, which will yield translation of the gene of interest. During translation of the leader peptide, any downstream mRNA structures involving the second RBS are dissolved, thus ensuring efficient translation of the desired gene, a proven and popular concept in synthetic biology. Previous research showed that the phi15 promoter can successfully be combined with the strong bicistronic 5′UTR BCD2, but expression levels are below those obtained with the native 5′UTR of the phi15 major capsid protein (MCP). Building on this knowledge, we attempted to combine the translational strength of the phage MCP 5′UTR with the standardized performance of BCD2 by creating novel BCDs based on the phages' MCP 5′UTR sequence. More specifically, we created five different BCDs for the phi15 system by ligating the phi15 promoter, phi15 MCP 5′UTR and the first 17 codons of the phi15 MCP gene, in which we introduced the second RBS and linker to the startcodon in five different ways (FIG. 27). All five phi15BCDs were ligated to an msfGFP reporter and introduced together with the phi15 RNAP in P. putida and their performance was compared to the original phi15 MCP 5′UTR and BCD2.

As displayed in FIG. 27C, the five phi15-based BCDs generate broadly varying msfGFP expression levels, ranging from 93.4 5(6)-FAM/OD₆₀₀ (phi15BCD02) to 335 5(6)-5(6)-FAM/OD₆₀₀ (phi15BCD05). The most important observation in this assay is that the msfGFP output of phi15BCD05 is significantly higher than the original phi15 MCP 5′UTR, showing a 41% increase (Tukey HSD, P<0.05). Therefore, this phi15-based BCD can be used as a highly potent 5′UTR in the optimized expression vector.

T7 gp2 Homologue Phi15 gp16 Inhibits the Host RNA Polymerase and Allows Growth Decoupling

In industrial set-ups, the concept of growth-decoupling is gaining popularity as it optimizes the use of resources in two phases, the growth phase and production phase, respectively. In the first phase, all resources go towards cell growth to acquire a healthy cell population. Next, cell growth is blocked and the production of the desired product is started, ensuring maximum use of the resources towards product formation. In E. coli, this concept has successfully been put into practice with the use of T7 gp2, an inhibitor of the host RNA polymerase. To recreate this concept in Pseudomonas, three different phage ORF (open reading frames) were cloned into pSTDesR, namely LUZ24 gp24 (Igy), a DNA gyrase inhibitor of P. aeruginosa, LUZ19 gp28 (Rac), a host RNAP inhibitor in P. aeruginosa and phi15 gp16, a homologue to T7 gp2 and a potential inhibitor of P. putida RNA polymerase. The phage ORFs were paired with phi15 RNAP and phi15 reporter construct in P. putida and the OD₆₀₀ and msfGFP output were monitored for 12 h post induction (FIG. 21). Both LUZ24 gp9 and LUZ19 gp28 do not impact the growth rate of P. putida, while phi15 gp16 halts cell growth after 4 h of induction with 10 mM Rha, after which the OD₆₀₀ remains stable at 1.4. At the same time, the msfGFP output of the strains expressing LUZ24 gp9 and LUZ19 gp28 is similar to the control, while the strain expressing phi15 gp16 shows a significant increase of ˜20% of msfGFP production (P<0.0001). These results prove that phi15 gp16 efficiently halts cell growth while still supporting metabolic activity in the form of msfGFP formation.

### Vector Backbone of Expression Vector

Multiple factors can influence the expression of the construct, such as the plasmid copy number and proper insulation from upstream and downstream sequences. So far, the msfGFP reporter constructs have been genomically integrated in the host as a single copy. To determine the impact of the copy number on msfGFP production, the reporter construct Pphi15, MCP-msfgfp is integrated in identical vectors with different origins of replication: pSEVA621 (RK2-low copy number), pSEVA631 (pBBR1-medium copy number), pSEVA641 (pR01600/ColE1-high copy number) and pSEVA651 (RSF1010-high copy number). When introducing the vectors in P. putida carrying pSTDesX.phi15RNAP none of the transformants contained the desired vector. This result is in line with the work of several other research groups, who did not succeed to support both a T7-like phage RNAP and the corresponding promoter on DNA vectors. Therefore, we attempted to introduce the vectors in P. putida where the phi15 RNAP is genomically integrated in PP13, PP5322 and PP5042, respectively. All of the strains were able to tolerate and replicate pSEVA621.Pphi15, MCP-msfGFP, while no transformants were obtained for the pSEVA641 backbone. Furthermore, only P. putida PP5042::phi15rnap was electroporated successfully with pSEVA631. Pphi15, MCP-msfGFP and pSEVA651. Pphi15, MCP-msfGFP.

The OD₆₀₀ and fluorescence intensity of all obtained transformants were measured for 12 h after induction with 0.3 mM 3 mBz and displayed in FIG. 25. For all integration sites, the cell cultures have a significantly lower OD₆₀₀ after 12 h of growth when the reporter construct vector-borne instead of integrated in the host's Tn7 landing site (P<0.0001). Moreover, for integration site PP13, almost no cell growth occurred after 12 h of incubation in the presence of 0.3 mM 3 mBz (FIG. 25). Due to this very stunted cell growth, it would be incorrect to compare the performance of the different strains based on their normalized msfGFP levels. Therefore, the absolute msfGFP levels were examined instead (FIG. 25), clearly indicating that a single copy reporter construct is highly favorable over multicopy variants, no matter in which locus the phi15 RNAP is integrated. For P. putida strains with phi15 RNAP integrated in PP13, PP5322 or PP5042, the single copy construct shows minimal impact on cell growth, while enabling very high absolute and normalized msfGFP expression levels. Based on these results, the pBGDes vector will be used as a backbone to create our final expression construct.

### Upstream Terminator

Nine strong, validated terminators of phages LUZ7 and LUZ100 were screened as potential upstream insulators of the expression construct. All terminators were individually placed directly upstream of the phi15 promoter driving msfGFP expression. Upon integration of the reporter constructs in the host' Tn7 attB site, the basal fluorescence level of the resulting strains was measured in absence of the phi15 RNAP, to assess potential readthrough of neighboring sequences. As a negative control, a reporter construct without upstream terminator and the P. putida KT2440 wildtype strain were included in the assay. The constructs with terminators LUZ7 T7 and LUZ7 T60 showed a msfGFP output that was significantly higher than the wildtype strain (Tukey HSD, P<0.05) (Error! Reference source not found.28A). It is unclear whether this increased msfGFP level is caused by the terminator sequence itself or another factor, but nevertheless these two terminators will not be considered as upstream insulators.

Apart from the basal fluorescence level, we confirmed that the tested terminators did not impact the msfGFP expression levels upon induction of the phi15 RNAP (Error! Reference source not found.28B). After introduction of the phi15 RNAP on pSTDesX, the msfGFP levels of all strains were monitored in induced and uninduced conditions. None of the terminators showed a significant impact on msfGFP output in comparison to a terminatorless control, except for LUZ100 T6 (Tukey HSD, P<0.05). Based on these results, terminator LUZ7 T50 is selected as an upstream insulator, as it does not influence the performance of the expression system and has been characterized as strong, bidirectional terminator.

### BCD Design

Building on this knowledge, we attempted to combine the translational strength of the phage MCP 5′UTR with the standardized performance of BCD2 by creating novel BCDs based on the phages' MCP 5′UTR sequence. More specifically, we created five different BCDs for the phi15 system by ligating the phi15 promoter, phi15 MCP 5′UTR and the first 17 codons of the phi15 MCP gene, in which we introduced the second RBS and linker to the startcodon in five different ways (FIG. 27A,B). In phi15BCD01, the MCP sequence was maximally maintained, while in phi15BCD02, phi15BCD03 and phi15BCD04 three RBS-startcodon spacers with different lengths were introduced that are reported to yield high expression levels in P. putida. Lastly, in phi15BCD05 we introduced the BCD2 spacer in the phi15 MCP. All five phi15BCDs were ligated to an msfGFP reporter and introduced together with the phi15 RNAP in P. putida strains pB4RB0, pB5RB0, pB6RB0, pB7RB0 and pB8RB0 (FIG. 27) and their performance was compared to the original phi15 MCP 5′UTR and the phi15 GC-GCAG-BCD2 UTR.

As displayed in FIG. 27C, the five phi15-based BCDs generate broadly varying msfGFP expression levels, ranging from 93.4 5(6)-FAM/OD₆₀₀ (phi15BCD02) to 335 5(6)-5(6)-FAM/OD₆₀₀ (phi15BCD05). Interestingly, phi15BCD05, phi15BCD04 and phi15BCD01 show the highest expression levels in the same range as the original phi15 MCP 5′UTR and all have a 7 nt spacer. This is in contrast to phi15BCD02 and phi15BCD03, which display much lower msfGFP expression levels and have a 9 nt and 8 nt spacer, respectively. This result was unexpected, as higher expression levels were reported for the 9 nt spacer than the shorter 8 nt spacer. This could be explained by early stop codons in the leader peptides of phi15BCD02 and phi15BCD03 (FIG. 27B), which often lead to lower expression levels in BCDs [56]. The most important observation in this assay is that the msfGFP output of phi15BCD05 is significantly higher than the original phi15 MCP 5′UTR, showing a 41% increase (P<0.05). Therefore, this phi15-based BCD can be used as a highly potent 5′UTR in the Pseudomonas-optimized pET system. To verify that the previous results are independent of the sequence of the gene of interest, a similar assay was performed in which msfGFP reporter was replaced with mScarlet-I, which have 43.9% overall sequence similarity on the DNA level (EMBOSS Needle). As shown in FIG. 27C, the expression levels of the phi15-based BCDs show the same order for both the msfGFP and mScarlet-I reporters, supporting the idea that BCD designs perform more independently from the gene's sequence than traditional monocistronic designs. Furthermore, phi15BCD05 shows a 13% increase of mScarlet-I expression levels, although this difference is not significant (P>0.05). Based on these results, we carefully conclude that phi15BCD05 is a reliable 5′UTR for use in genetic circuitry.

### Downstream Terminator

Efficient transcription termination is known to play a crucial role in circuit performance. Therefore, eleven phage terminators were screened for efficient transcription termination of the phi15 RNAP using the SEVAtile terminator trap (Error! Reference source not found). In this trap, a terminator is placed in between msfGFP and mCherry, such that low mCherry are indicative for transcriptional termination, while the msfGFP output can be related to increased or decreased mRNA stability by the terminator sequence. The terminator efficiency of all tested terminators was significantly higher than the terminator-less control construct (P<0.05) (Error! Reference source not found). Moreover, terminators phi15 T1 and phi15 T5 outperformed all other terminators with an efficiency of 96.1% and 94.5%, respectively. Interestingly, not only was the normalized mCherry level of the phi15 T1 sample 15-fold lower than the control, the msfGFP levels were also twice as high compared to the control (Error! Reference source not found).

To increase the termination even more, terminators phi15 T1 and phi15 T5 were placed in tandem in both possible orders. For the phi15 T5+T1 construct, a terminator efficiency of 99.5% was observed, which was 2% higher than the phi15 T1+T5 combination. As such, the terminator pair phi15 T5+T1 is selected for placement downstream of the reporter construct, to efficiently terminate the phi15 RNAP.

### EXAMPLES

### Example 1. Materials and Methods

### Bacteriophage Genomes

All bacteriophage sequences used in the present invention originated from phages that were isolated, sequenced, and annotated in previous research, as shown in Table 2.

TABLE 2
List of bacteriophage genomes used in the present invention.

| Bacteriophage | Accession Number | Reference |
|---|---|---|
| Phi15 | FR823298.1 | Cornelissen et al. |
| Pf-10 | NC_027292.1 | Unpublished |
| PPPL-1 | NC_028661.1 | Park et al. |
| 67PfluR64PP | MH179478.2 | Kazimierczak et al. |

Cornelissen (2011) PLoS ONE 6, e18597; Park et al. (2018) J. Microbiol. Biotechnol. 28, 1542-1546; Kazimierczak et al. (2019) Virol. J. 16, 4.

### Bacterial Manipulation

In this study, two E. coli strains were used for vector cloning purposes, i.e., E. coli TOP10 as a main host and E. coli PIR2 for pBGDes-derived vectors carrying the R6K origin. The characterization and optimization of phage-based elements were performed in P. putida KT2440 or P. aeruginosa PAO1. All strains were cultured overnight in a sterile LB medium or LB agar, supplemented with antibiotics as required: Amp¹⁰⁰, Kan⁵⁰, Gm¹⁰ (E. coli and P. putida) or Gm³⁰ (P. aeruginosa), Tc¹⁰ (E. coli and P. putida) or Tc⁶⁰ (P. aeruginosa), and Sp⁵⁰ and Sm²⁰⁰. E. coli and P. aeruginosa were incubated at 37° C., whereas P. putida was standardly incubated at 30° C. Plasmid vectors were introduced in all strains by transformation. E. coli was transformed using rubidium chloride, whereas P. putida and P. aeruginosa were electroporated. The pBGDes vectors were always co-electroporated with a helper plasmid, pTNS2, to ensure genomic integration of pBGDes in the Tn7 attB site of the host.

### Vector Construction

To screen the selected phage RNAPs, promoters, and lysozymes to create a tailored pET system for P. putida, the SEVAtile vector set was used, which enables rapid and standardized assembly of genetic circuits. As a positive control, the T7-based pET system was recreated with the SEVAtile vectors in P. putida and P. aeruginosa, as shown previously, with the T7 RNAP in pSTDesX, the T7 lysozyme in pSTDesR, and a reporter construct with P_{T7,MCP}-msfGFP integrated into the Tn7 attB site using pBGDes. To screen phage RNAPs, promoters, and lysozymes from T7-related phages in a similar setup, all necessary vectors were assembled using SEVAtile-shuffling by first amplifying the phage-encode parts with a tail-PCR to add the required overhangs for SEVAtile-shuffling. Gibson assembly was used for vector assembly in case the phage genes contained one or multiple BsaI recognition sites. Assembled vectors were introduced in E. coli TOP10 or E. coli PIR2 for pBGDes-derived vectors, and the correct insertion of phage-encoded genes was verified by Sanger sequencing (Eurofins Genomics, Ebersberg, Germany).

### Toxicity Evaluation by Growth Curve Monitoring

To assess the potential cytotoxicity of recombinantly expressed phage RNAPs and lysozymes on P. putida and P. aeruginosa, 12 h growth curves of all relevant cultures were prepared. First, overnight cultures of four biological replicates were diluted 20-fold in a fresh growth medium in a 96-well plate with the appropriate antibiotics and incubated for 3 h while shaking at the appropriate temperature. At this time point, every cell culture was split in an uninduced and induced fraction by adding the appropriate inducer to the latter. For RNAP toxicity, a final concentration of 1 mM 3-methylbenzoate (3-mBz) was introduced, while for lysozyme toxicity, 10 mM L-rhamnose was supplied to the culture. Culture plates were directly placed in a CLARIOstar® Plus Microplate Reader (BMG Labtech, Ortenberg, Germany), where OD₆₀₀ measurements were performed every 30 min for a total period of 12 h while incubating at the appropriate temperature with intermittent shaking. The resulting data were corrected for blank values (sterile growth medium) and statistically analyzed using JMP 16 Pro (JMP®, Version 16. SAS Institute Inc., Cary, NC, USA, 1989-2021). Multiple comparisons of the mean values were performed on the final timepoint by first confirming the normality of the data for each sample (Shapiro-Wilk test, α=0.05), followed by the Tukey HSD (honest significant difference) test, with correction for multiple comparisons (α=0.05).

### Fluorescence Intensity Assays

To verify the performance of the phage elements in both P. putida KT2440 and P. aeruginosa PAO1, fluorescent expression assays were performed. Overnight cultures of four biological replicates of P. putida KT2440 or P. aeruginosa PAO1 carrying the appropriate vectors were prepared in an M9 minimal medium containing 1×M9 salts (BD Biosciences, Franklin Lakes, NJ, USA), 0.2% citrate (Sigma Aldrich, St. Louis, MO, USA), 2 mM MgSO₄ (Sigma Aldrich), 0.1 mM CaCl₂) (Sigma Aldrich), 0.5% casein amino acids (LabM; Neogen® Company, Lansing, MI, USA), and the appropriate antibiotics. Each overnight culture was diluted 20-fold in a fresh M9 medium in a Corning®) 96-Well Black Polystyrene Microplate with a Clear Flat Bottom and incubated for 2 h in shaking conditions. At this point, the cell cultures were split in two to create an uninduced and induced sample, to which the required inducer(s) was added. Next, the fluorescence intensity and OD 600 levels were monitored every 30 min for 12 h on a CLARIOstar®) Plus Microplate Reader while incubating at 30° C. or 37° C. for P. putida KT2440 or P. aeruginosa PAO1, respectively. The fluorescent intensity of the msfGFP was measured at a 485 nm excitation wavelength and 528 nm emission wavelength with the enhanced dynamic range setting of the apparatus. All relative fluorescent measurements were blank-corrected for a sterile medium and normalized for cell growth by dividing by the corresponding OD₆₀₀ value. To convert the relative fluorescence units of the msfGFP to absolute units, a calibration curve was added to each experiment. More specifically, 0, 375, 750, and 1500 nM of 5(6)-carboxyfluorescein (5(6)-FAM) (Sigma Aldrich) in phosphate-buffered saline was added to each plate in duplicate. All (normalized) fluorescent measurements of the msfGFP were subsequently converted to the equivalent 5(6)-FAM concentration. The data were analyzed, visualized, and verified for statistical significance using JMP 16 Pro. Statistical significance assays were performed on the final timepoint by first confirming the normality of the data for each sample (Shapiro-Wilk), followed by an appropriate mean (multiple) comparisons test. In particular, if the data were normally distributed, a (pairwise) Student's t-test (α=0.05) was performed, while for non-normally distributed data, a (pairwise) Wilcoxon assay (α=0.05) was employed. No corrections for multiple comparisons were made due to large differences in variance between samples, except for the lysozyme optimization assays for inhibition of the phi15 RNAP, where variances were equal (Tukey HSD test, α=0.05).

Transcription Start Site Determination with 5′-Capping-RACE

The transcription start site (TSS) of each phage promoter was determined using 5′-capping-RACE (Rapid Amplification of cDNA Ends). First, the total RNA fraction of P. putida strains, pA0RA0, pB0RB0, pC0RC0, pD0RD0, and pE0RE0, was harvested as follows. Overnight cultures were diluted 100-fold in a fresh LB medium with appropriate antibiotics and incubated at 30° C. in shaking conditions. Once the cells reached OD₆₀₀0.3, cultures were induced with 0.3 mM 3 mBz and incubated for 3 h upon harvesting at OD_{600 4}. The harvested cells were subjected to hot phenol/lysozyme to extract total RNA, followed by DNase I treatment. Next, CDNA was generated with the primers listed in Table 2. The resulting cDNA products were cloned into pSTEntry with SapI restriction-ligation and transformed to E. coli TOP10. Five transformants of each sample were treated with a GeneJET Plasmid Miniprep kit (Thermo Scientific) to isolate the pSTEntry.phage-cDNA vectors and Sanger sequenced with SEVA_PS1 and SEVA_PS2 primers.

TABLE 2
Primers used to determine the transcription start site of phage promoters
with 5′-capping-RACE.

| Name | Sequence |
|---|---|
| TSS_TSO | ACACTCTTTCCCTACACGACGCTCTTCCGATCTrGrGrG [SEQ ID NO: 6] |
| TSS_outerprimer | AATGATACGGCGACCACCGAGATCTACACTCTTTCCCTACACGACGCTC |
| | TTCCGATCT [SEQ ID NO: 7] |
| TSS_innerprimer | ATAGCTCTTCTAGACTACACGACGCTCTTCCGATCT [SEQ ID NO: 8] |
| TSS_GSP1 | TCAGTTTACCGTTGGTTGCATCACCTTCACCTTCACCACGAACAGAGAA |
| | TTTGTGGCC [SEQ ID NO: 9] |
| TSS_GSP2 | TAGGCTCTTCTCTTCGAACAGAGAATTTGTGGCC [SEQ ID NO: 10] |

### Flow Cytometry

Single-cell fluorescent data of strains were obtained by flow cytometry. First, overnight cultures were prepared in duplo, which was diluted 20-fold in a fresh M9 medium the following day in a clear 96-well plate with a flat bottom and incubated for 2 h in shaking conditions. At this point, the cell cultures were split in two to create an uninduced and induced sample, to which the required inducer(s) was added. After overnight induction, samples were diluted tenfold in 200 μL of a PBS medium (pH 7.4, filter-sterilized (0.22 μm)) and analyzed on a CytoFLEXS® Flow Cytometry machine (Beckman, San Jose, CA, USA). Then, 5000 events (i.e., individual cells) were screened for FSC-A (gain 165), SSC-A (gain 400), and FITC-A (gain 10), with a maximal flow rate of 1000 events/μL. The FITC-A channel detected msfGFP fluorescence of single cells, where a value above 10⁴ was considered positive for msfGFP fluorescence.

TABLE 3
Connecting letters report of a pairwise Student's t-test
of the cross-recognition assay between phage promoters and
RNAPs. Levels not connected by same letter are different.

| Promoter | RNAP | | | | | | Mean |
|---|---|---|---|---|---|---|---|
| T7 | T7 | A | | | | | 2060.566 |
| phi15 | phi15 | | B | | | | 660.3784 |
| PPPL-1 | PPPL-1 | | | C | | | 245.7189 |
| Pf-10 | Pf-10 | | | C | D | | 189.0884 |
| T7 | phi15 | | | | D | E | 85.7572 |
| 67PfluR64PP | T7 | | | | | E | 41.8903 |
| T7 | PPPL-1 | | | | | E | 40.0312 |
| Pf-10 | PPPL-1 | | | | | E | 36.1123 |
| PPPL-1 | T7 | | | | | E | 29.5002 |
| Pf-10 | phi15 | | | | | E | 29.4009 |
| Pf-10 | 67PfluR64PP | | | | | E | 25.9146 |
| T7 | 67PfluR64PP | | | | | E | 22.6429 |
| 67PfluR64PP | 67PfluR64PP | | | | | E | 22.1996 |
| T7 | Pf-10 | | | | | E | 21.0388 |
| PPPL-1 | phi15 | | | | | E | 15.8159 |
| 67PfluR64PP | Pf-10 | | | | | E | 15.3239 |
| phi15 | T7 | | | | | E | 12.4631 |
| PPPL-1 | Pf-10 | | | | | E | 10.3922 |
| Pf-10 | T7 | | | | | E | 8.5055 |
| 67PfluR64PP | phi15 | | | | | E | 7.5807 |
| phi15 | 67PfluR64PP | | | | | E | 7.239 |
| PPPL-1 | 67PfluR64PP | | | | | E | 7.1203 |
| 67PfluR64PP | PPPL-1 | | | | | E | 3.9164 |
| phi15 | PPPL-1 | | | | | E | 2.1663 |
| phi15 | Pf-10 | | | | | E | 2.134 |

1-35. (canceled)

36. A Pseudomonas sp. strain comprising a nucleotide sequence encoding a phi15 RNA polymerase.

37. The Pseudomonas sp. strain according to claim 36, wherein the phi15 RNA polymerase is under control of an inducible promoter.

38. The Pseudomonas sp. strain according to claim 37, wherein the inducible promoter is XylS/pM promoter/regulator.

39. The Pseudomonas sp. strain according to claim 36, wherein the phi15 RNA polymerase has a protein sequence of accession number YP_004286187.1.

40. The Pseudomonas sp. strain according to claim 39, wherein the phi15 RNA polymerase comprises a R630S mutation relative to the protein sequence of accession number YP 004286187.1.

41. The Pseudomonas sp. strain according to claim 37, wherein the inducible promoter is XylS/pM promoter/regulator.

42. The Pseudomonas sp. strain according to claim 36, wherein the nucleotide sequence encoding the phi15 RNA polymerase is integrated into the genome of the Pseudomonas sp. strain.

43. The Pseudomonas sp. strain according to claim 42, wherein the nucleotide sequence encoding the phi15 RNA polymerase is integrated in a PP13 (gyrB) locus of the Pseudomonas sp. strain.

44. The Pseudomonas sp. strain according to claim 36, wherein the Pseudomonas sp. strain is Pseudomonas putida.

45. The Pseudomonas sp. strain according to claim 36, wherein the strain comprises a weak ribosome binding sequence selected from SEQ ID NO: 1 or SEQ ID NO: 2.

46. The Pseudomonas sp. strain according to claim 36, further comprising a nucleotide sequence encoding a phi15 lysozyme.

47. The Pseudomonas sp. strain according to claim 46, wherein the nucleotide sequence encoding the phi15 lysozyme is integrated into the genome of the Pseudomonas sp. strain.

48. The Pseudomonas sp. strain according to claim 47, wherein the phi15 lysozyme is integrated in a PP4305 locus of the genome of Pseudomonas putida strain KT2440.

49. The Pseudomonas sp. strain according to claim 46, wherein the phi15 lysozyme is comprised in a plasmid vector.

50. The Pseudomonas sp. strain according to claim 46, wherein the phi15 lysozyme is under control of an inducible promoter comprising a RhaRS/P_{rhaBAD} promotor/regulator.

51. The Pseudomonas sp. strain according to claim 46, wherein the phi15 lysozyme is under control of a constitutive promoter comprising p14c.

52. The Pseudomonas sp. strain according to claim 46, wherein the phi15 lysozyme has a protein sequence of accession number YP_004286199.1 or has a G3RQ mutation relative to the protein sequence of accession number YP_004286199.1, wherein glycine at position 3 is replaced by the dipeptide arginine-glutamine.

53. The Pseudomonas sp. strain according to claim 46, wherein the phi15 lysozyme sequence comprises a BCD22 ribosome binding site.

54. The Pseudomonas sp. strain according to claim 46, further comprising a nucleotide sequence encoding a phi15 GP16 RNA polymerase inhibitor having the sequence of accession number YP_004286194.1.

55. The Pseudomonas sp. strain according to claim 36, further comprising a plasmid capable of integrating or replicating in the Pseudomonas sp. strain, the plasmid comprising a phi15 promoter sequence operably linked to:
a nucleotide comprising one or more restriction sites for insertion of a nucleotide sequence encoding a recombinant protein; or
a nucleotide sequence encoding the recombinant protein.

56. A method of producing a recombinant protein, the method comprising administering an agent that induces transcription and translation of the phi15 RNA polymerase to the Pseudomonas sp. strain of claim 55 comprising the nucleotide sequence encoding the recombinant protein.


SEQUENCE LISTING (53 sequences)

SEQ ID NO: 1 - DNA, 36 bp, synthetic construct
  source 1..36: mol_type = other DNA
taaagctttatctatttaacaacgcggtccgatgtg

SEQ ID NO: 2 - DNA, 36 bp, synthetic construct
  source 1..36: mol_type = other DNA
taaagctttatctatttaacaacggggtccgaggtg

SEQ ID NO: 3 - DNA, 117 bp, synthetic construct
  source 1..117: mol_type = other DNA
  CDS 62..115: protein_id = 4; translation = MATLNNGTKQGQNKEVF
gacagagggagccgttccgtgctccttctggacccaatttctgactcaaggagaactacatatggcaactctgaacaacggcaccaagcaaggccagaataaggaggttttctaatg

SEQ ID NO: 4 - protein, 17 aa, synthetic construct
MATLNNGTKQGQNKEVF

SEQ ID NO: 5 - DNA, 17 bp, synthetic construct
  source 1..17: mol_type = other DNA
taaaaacccacacaata

SEQ ID NO: 6 - DNA, 39 bp, synthetic construct
  source 1..39: mol_type = other DNA
acactctttccctacacgacgctcttccgatctrgrgrg

SEQ ID NO: 7 - DNA, 58 bp, synthetic construct
  source 1..58: mol_type = other DNA
aatgatacggcgaccaccgagatctacactctttccctacacgacgctcttccgatct

SEQ ID NO: 8 - DNA, 36 bp, synthetic construct
  source 1..36: mol_type = other DNA
atagctcttctagactacacgacgctcttccgatct

SEQ ID NO: 9 - DNA, 58 bp, synthetic construct
  source 1..58: mol_type = other DNA
tcagtttaccgttggttgcatcaccttcaccttcaccacgaacagagaatttgtggcc

SEQ ID NO: 10 - DNA, 34 bp, synthetic construct
  source 1..34: mol_type = other DNA
taggctcttctcttcgaacagagaatttgtggcc

SEQ ID NO: 11 - protein, 882 aa, Pseudomonas phage phi15
MIEVAKNDFSDVKTDWAFRVLSELYGEELAAAQLALEHESHEIGEAKFKKALDRQMKRGETSETSVAKPLVAMLVPKFVEKMDAWVEHQMKNVRRKSVALKFIQMVATERVAVITIKTVINAMSQGDVVLQAIAGRIGRGIEEEARFGRIRDQEAKHFKKYIREALNKRNGHTYKRAYMHAVEDRMLEAGELNGAWSDWDNEDPTIIAHIGLRCIEALIESSGLVRITRRSAGNVKEDCNVLELEPQWVEMLNQRAFTLAGVNTYHQPCVVPPRPWTRPVGGGYWGKGRRPTRFIRVHNKKALERYRDVDMEAVYKAVNIAQNTAWSINKRILEVAEALASWTNVPISKWPKAETQELPVKPHDIETNEEARNAWKKQASGVYRSESSRVSRRMSLETTLETARKFADFEAIYFPHNLDWRGRVYALPVFNPQRDDLTKGLLQASKGEPVGEDGIKWLMIHGANTAGVDKVPFDERQQWVRDNERTILQCAEDPLTHTEWMSMDSPFCFLAFCFEWAGVVKDGPNHVSALPIAFDGSCSGIQHFSAMLRDETGGRAVNLLPSERVQDIYRLVSDGVNAALRDDAVHGTDDSTDVHVDEKTGEITERRVLGTRTLAAAWLAHGVDRSVTKRSVMTLAYGSKEFGFTDQVRDDIITPAVDAGSLNFPQPQQAARYMAHLIWVSVGKTVVAAVEAMEWLQKSAKLLAAIVKEKKGPEKGKILKPAMPVYWVTPDGFPVWQEYRVQQAKRIDMILMGDVRLTATVLHQQDQIDARKQESGISPNFVHSMDGNHLRQTVVHAHDAYDITFFALIHDSFGTIPAKAGQLFKAVRETMVTAYEHNDVLADFREQFIDQLHETQMDKMPELPKKGTLDIREILKSQFAFA

SEQ ID NO: 12 - protein, 882 aa, Pseudomonas phage phi15
MIEVAKNDFSDVKTDWAFRVLSELYGEELAAAQLALEHESHEIGEAKFKKALDRQMKRGETSETSVAKPLVAMLVPKFVEKMDAWVEHQMKNVRRKSVALKFIQMVATERVAVITIKTVINAMSQGDVVLQAIAGRIGRGIEEEARFGRIRDQEAKHFKKYIREALNKRNGHTYKRAYMHAVEDRMLEAGELNGAWSDWDNEDPTIIAHIGLRCIEALIESSGLVRITRRSAGNVKEDCNVLELEPQWVEMLNQRAFTLAGVNTYHQPCVVPPRPWTRPVGGGYWGKGRRPTRFIRVHNKKALERYRDVDMEAVYKAVNIAQNTAWSINKRILEVAEALASWTNVPISKWPKAETQELPVKPHDIETNEEARNAWKKQASGVYRSESSRVSRRMSLETTLETARKFADFEAIYFPHNLDWRGRVYALPVFNPQRDDLTKGLLQASKGEPVGEDGIKWLMIHGANTAGVDKVPFDERQQWVRDNERTILQCAEDPLTHTEWMSMDSPFCFLAFCFEWAGVVKDGPNHVSALPIAFDGSCSGIQHFSAMLRDETGGRAVNLLPSERVQDIYRLVSDGVNAALRDDAVHGTDDSTDVHVDEKTGEITERRVLGTRTLAAAWLAHGVDRSVTKSSVMTLAYGSKEFGFTDQVRDDIITPAVDAGSLNFPQPQQAARYMAHLIWVSVGKTVVAAVEAMEWLQKSAKLLAAIVKEKKGPEKGKILKPAMPVYWVTPDGFPVWQEYRVQQAKRIDMILMGDVRLTATVLHQQDQIDARKQESGISPNFVHSMDGNHLRQTVVHAHDAYDITFFALIHDSFGTIPAKAGQLFKAVRETMVTAYEHNDVLADFREQFIDQLHETQMDKMPELPKKGTLDIREILKSQFAFA

SEQ ID NO: 13 - protein, 153 aa, Pseudomonas phage phi15
MAGVKFKERLSTKMIVVHCSATKASMDIGRKEIQMWHVQQGWLAIGYHLVIRRDGTIEQGRPHKAIGSHVKGHNSDSIGICLVGGIDDSGKPEDNFTDQQKAALSGLLWDMTQSGVTYGDTYKELPVVGHRDLDSGKACPSFDVKAWWAAQIN

SEQ ID NO: 14 - protein, 154 aa, Pseudomonas phage phi15
MARQVKFKERLSTKMIVVHCSATKASMDIGRKEIQMWHVQQGWLAIGYHLVIRRDGTIEQGRPHKAIGSHVKGHNSDSIGICLVGGIDDSGKPEDNFTDQQKAALSGLLWDMTQSGVTYGDTYKELPVVGHRDLDSGKACPSFDVKAWWAAQIN

SEQ ID NO: 15 - protein, 56 aa, Pseudomonas phage phi15
MNVEKQKMKRFEAELVGKFTSSWVPVWARNVDEALEAAEMEYGEDNVGRVRPKEVE

SEQ ID NO: 16 - DNA, 100 bp, synthetic construct
  source 1..100: mol_type = other DNA
ttctgggagatcctggcaaacgcattcaaaaagaaaggggagcattagctccccttcttctttacaggttcatcaaacctttcaccatcggcagttcact

SEQ ID NO: 17 - DNA, 101 bp, synthetic construct
  source 1..101: mol_type = other DNA
gtggttaccgttcgataagctgaaaccaaaccccttgggtcccataacggggcttgaggggtttttatttagaacaggaggagctatggctctacaagatg

SEQ ID NO: 18 - DNA, 100 bp, synthetic construct
  source 1..100: mol_type = other DNA
gcgtggccatcttcgggagttaagtccactgaagggcatcctcatgggtgccttttggggaacaaatacccacactatcgggaatatcgaaatgaaatac

SEQ ID NO: 19 - DNA, 83 bp, synthetic construct
  source 1..83: mol_type = other DNA
taatacgactcactatagggagaccacaacggtttccctctagaaataattttgtttaactttaagaaggagatatacatatg

SEQ ID NO: 20 - DNA, 82 bp, synthetic construct
  source 1..82: mol_type = other DNA
ttaaaaaccctcacctaaacagggaggaccattttggttcctctctgaacacttttcgattacataaggagtcccacaaatg

SEQ ID NO: 21 - DNA, 101 bp, synthetic construct
  source 1..101: mol_type = other DNA
taaaacccctcactattgcgacacacacggaccttcaggcaaatcctgagggttgactgtgcgtgtccttcaagaatctcaaagaaggagaactacacatg

SEQ ID NO: 22 - DNA, 83 bp, synthetic construct
  source 1..83: mol_type = other DNA
taaaaaccctcacctatacaggagggagccactaaggttccttcttgatcccatttgatttgcataaggagttccactacatg

SEQ ID NO: 23 - DNA, 81 bp, synthetic construct
  source 1..81: mol_type = other DNA
taaaaacccacacaatagacagagggagccgttccgtgctccttctggacccaatttctgactcaaggagaactacatatg

SEQ ID NO: 24 - DNA, 46 bp, synthetic construct
  source 1..46: mol_type = other DNA
agcgagagcttcagacctgatccaaattgctccagcgatcacgaag

SEQ ID NO: 25 - DNA, 46 bp, synthetic construct
  source 1..46: mol_type = other DNA
ggcgctcgattctgtggcgatgccgagctgggcatgaagcactgaa

SEQ ID NO: 26 - DNA, 46 bp, synthetic construct
  source 1..46: mol_type = other DNA
tgctgaagctggattgatggcccaatcggtcaggggttgccggcag

SEQ ID NO: 27 - DNA, 36 bp, synthetic construct
  source 1..36: mol_type = other DNA
taatggagtcatgaccatgaggaggaaaaacaaatg

SEQ ID NO: 28 - DNA, 43 bp, synthetic construct
  source 1..43: mol_type = other DNA
ctataccggtcagtaacgcacaagcaacatcaaccccggcgga

SEQ ID NO: 29 - DNA, 54 bp, synthetic construct
  source 1..54: mol_type = other DNA
  CDS 1..54: protein_id = 30; translation = MKAIFVLKHLNHAKEVF
atgaaagcaattttcgtactgaaacatcttaatcatgctaaggaggttttctaa

SEQ ID NO: 30 - protein, 17 aa, synthetic construct
MKAIFVLKHLNHAKEVF

SEQ ID NO: 31 - DNA, 54 bp, synthetic construct
  source 1..54: mol_type = other DNA
  CDS 1..54: protein_id = 32; translation = MATLNNGTKQGQNQEEG
atggcaactctgaacaacggcaccaagcaaggccagaaccaggaggagggctaa

SEQ ID NO: 32 - protein, 17 aa, synthetic construct
MATLNNGTKQGQNQEEG

SEQ ID NO: 33 - DNA, 54 bp, synthetic construct
  source 1..54: mol_type = other DNA
  CDS 1..39: protein_id = 34; translation = MATLNNGTKQGQ
atggcaactctgaacaacggcaccaagcaaggccagtaaggaggtaaaaactaa

SEQ ID NO: 34 - protein, 12 aa, synthetic construct
MATLNNGTKQGQ

SEQ ID NO: 35 - DNA, 54 bp, synthetic construct
  source 1..54: mol_type = other DNA
  CDS 1..48: protein_id = 36; translation = MATLNNGTKQGQIRR
atggcaactctgaacaacggcaccaagcaaggccagataaggaggtaaaactaa

SEQ ID NO: 36 - protein, 15 aa, synthetic construct
MATLNNGTKQGQIRR

SEQ ID NO: 37 - DNA, 54 bp, synthetic construct
  source 1..54: mol_type = other DNA
  CDS 1..54: protein_id = 38; translation = MATLNNGTKQGQNKEVN
atggcaactctgaacaacggcaccaagcaaggccagaataaggaggtaaactaa

SEQ ID NO: 38 - protein, 17 aa, synthetic construct
MATLNNGTKQGQNKEVN

SEQ ID NO: 39 - DNA, 54 bp, synthetic construct
  source 1..54: mol_type = other DNA
  CDS 1..54: protein_id = 40; translation = MATLNNGTKQGQNKEVF
atggcaactctgaacaacggcaccaagcaaggccagaataaggaggttttctaa

SEQ ID NO: 40 - protein, 17 aa, synthetic construct
MATLNNGTKQGQNKEVF

SEQ ID NO: 41 - DNA, 68 bp, synthetic construct
  source 1..68: mol_type = other DNA
taatacgactcactatagggcaggcccaagttcacttaaaaaggagatcaacaatgaaagcaattttc

SEQ ID NO: 42 - DNA, 80 bp, synthetic construct
  source 1..80: mol_type = other DNA
taatacgactcactatagggagaccacaacgcaggcccaagttcacttaaaaaggagatcaacaatgaaagcaattttcg

SEQ ID NO: 43 - DNA, 17 bp, synthetic construct
  source 1..17: mol_type = other DNA
taatacgactcactata

SEQ ID NO: 44 - DNA, 17 bp, synthetic construct
  source 1..17: mol_type = other DNA
taaaaacccacacaata

SEQ ID NO: 45 - DNA, 17 bp, synthetic construct
  source 1..17: mol_type = other DNA
taaaacccctcactatt

SEQ ID NO: 46 - DNA, 18 bp, synthetic construct
  source 1..18: mol_type = other DNA
ttaaaaaccctcacctaa

SEQ ID NO: 47 - DNA, 16 bp, synthetic construct
  source 1..16: mol_type = other DNA
aaaaaccctcacctat

SEQ ID NO: 48 - DNA, 91 bp, synthetic construct
  source 1..91: mol_type = other DNA
ctacacgacgctcttccgatctggggggagaccacaacggtttccctctagaaataattttgtttaactttaagaaggagatatacaaatg

SEQ ID NO: 49 - DNA, 91 bp, synthetic construct
  source 1..91: mol_type = other DNA
ctacacgacgctcttccgatctggggacagagggagccgttccgtgctccttctggacccaatttctgactcaaggagaactacaaatgat

SEQ ID NO: 50 - DNA, 91 bp, synthetic construct
  source 1..91: mol_type = other DNA
ctacacgacgctcttccgatctggggcgacacacacggaccttcaggcaaatcctgagggttgactgtgcgtgtccttcaagaatctcaaa

SEQ ID NO: 51 - DNA, 91 bp, synthetic construct
  source 1..91: mol_type = other DNA
ctacacgacgctcttccgatctggggacagggaggaccattttggttcctctctgaacacttttcgattacataaggagtcccacaaatga

SEQ ID NO: 52 - DNA, 91 bp, synthetic construct
  source 1..91: mol_type = other DNA
ctacacgacgctcttccgatctgggacaggagggagccactaaggttccttcttgatcccatttgatttgcataaggagttccactaaatg

SEQ ID NO: 53 - DNA, 44 bp, synthetic construct
  source 1..44: mol_type = other DNA
atctaggaagtcagggcgattcagatcatggtaaccccggccgc